SEQUENCE LISTING (12 sequences)

SEQ ID NO: 1 - DNA, 30 bp, Artificial Sequence
  Synthentic primer
aagcagtggtatcaacgcagagtacgcggg

SEQ ID NO: 2 - DNA, 25 bp, Artificial Sequence
  Synthentic primer
aagcagtggtatcaacgcagagtac

SEQ ID NO: 3 - DNA, 23 bp, Artificial Sequence
  Synthentic primer
aagcagtggtatcaacgcagagt

SEQ ID NO: 4 - DNA, 22 bp, Artificial Sequence
  Synthentic primer
gawyyayyhragccyaaadatg

SEQ ID NO: 5 - protein, 71 aa, Artificial Sequence
  Synthentic peptide
MFTLKKCMLLIFFLGTINLSLCQEESNAEEERRDDDDDQMNVEVEKRFFPGIIKVASAILPTAICAITKRC

SEQ ID NO: 6 - protein, 71 aa, Artificial Sequence
  Synthentic peptide
MFTLKKPLLLIFFLGTINLSLCQEESNAEEERRDDDDDQMNVEVEKRFFPGIIKVAGAILPTAICAITKRC

SEQ ID NO: 7 - protein, 24 aa, Artificial Sequence
  Synthentic peptide
FFPGIIKVASAILPTAICAITKRC

SEQ ID NO: 8 - protein, 24 aa, Artificial Sequence
  Synthentic peptide
FFPGIIKVASAILPTAICAITKRC

SEQ ID NO: 9 - protein, 24 aa, Artificial Sequence
  Synthentic peptide
FFPGIIKVASAILPTAICAITKRC

SEQ ID NO: 10 - protein, 24 aa, Artificial Sequence
  Synthentic peptide
FFPGIIKVAGAILPTAICAITKRC

SEQ ID NO: 11 - protein, 24 aa, Artificial Sequence
  Synthentic peptide
FFPGIIKVAGAILPTAICAITKRC

SEQ ID NO: 12 - protein, 24 aa, Artificial Sequence
  Synthentic peptide
FFPGIIKVAGAILPTAICAITKRC

(19) United States
(12) Patent Application Publication
(10) Pub. No.: US 2019/0298796 A1
(43) Pub. Date: Oct. 3, 2019

(54) Therapeutic Compositions from the Brevinin-1 Family of Peptides and Uses Thereof

(71) Applicant: Rajiv Gandhi Centre for Biotechnology, an Autonom- ous Institute under the Department of Biotechnolo, Thiruvananthapuram (IN)

(72) Inventors: Sanil George, Trivandrum (IN); T.V Vineethkumar, Cochin (IN)

(21) Appl. No.: 16/307,437

(22) PCT Filed: Jun. 15, 2017
(86) PCT No.: PCT/IN2017/050239

§ 371 Date: Dec. 5, 2018

(30) Foreign Application Priority Data
Jun. 15, 2016 (IN) 201641020429

(51) Int. Cl. A61K 38/17; A61K 45/06; C07K 14/46; C07K 1/00; A61P 31/04

(52) U.S. Cl. CPC A61K 38/1703; A61K 45/06; A61K 38/00; C07K 1/006; A61P 31/04; C07K 14/463

(57) ABSTRACT

The invention is directed to peptides and methods of making and using antimicrobial compositions for the treatment of a bacterium, wherein the composition comprises: a pharmaceutically effective amount of a modified brevinin-1 peptide, as well as modified and truncated versions thereof, disposed in a pharmaceutical carrier.

### CROSS-REFERENCE TO RELATED APPLICATIONS

None.

### TECHNICAL FIELD OF THE INVENTION

The present invention relates generally to methods and compositions used to treat bacterial infections and more specifically to brevinin-1 peptide as well as modified and truncated versions thereof for the treatment of a bacterium.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with new polypeptides for therapeutic use and their functional derivatives and pharmaceutically acceptable salts. The rise of drug-resistant pathogens that cause difficult-to-cure infections and the problem is particularly serious in the case of AIDS, tuberculosis and other immunocompromised patients. For example, it is estimated that more than 31% of bacterial isolates of Streptococcus pneumoniae from patients at U.S. hospitals were intermediately or completely resistant to penicillin and 29% of bacterial isolates of Streptococcus aureus were intermediately or completely resistant to methicillin. This emergence of increasing numbers of pathogenic microorganisms with resistance to the commonly used antibiotics has greatly stimulated searches for novel antimicrobial agents to fight drug-resistant infections. Among those searches is the investigation of novel antibiotic peptides from amphibians because they live in a warm, moist environment that is particularly conducive to the growth of pathogens, resulting in an evolutionarily need for protection. For example frog skin secretions contain many different types of antibacterial peptides.

For example, U.S. Pat. No. 6,310,176, entitled “Antimicrobially active polypeptides,” discloses a polypeptide selected from peptides and functional derivatives and pharmaceutically acceptable salts thereof; pharmaceutical compositions containing one or more of these polypeptides; and a method for inhibiting microbial growth in animals using such polypeptides.

### SUMMARY OF THE INVENTION

The present invention provides a cDNA composition encoding a peptide for reducing a bacterial population, wherein the cDNA composition comprises: an isolated cDNA encoding a brevinin-1 HYba1 peptide, a brevinin-1 HYba2 peptide or both.

The present invention provides an antimicrobial composition for the treatment of a bacterium, wherein the composition comprises: a pharmaceutically effective amount of a modified brevinin-1 peptide disposed in a pharmaceutical carrier.

The present invention provides a modified brevinin-1 peptide composition for use as a medicament for the treatment of a bacterial infection wherein the composition comprises: a pharmaceutically effective amount of modified brevinin-1 peptide disposed in a pharmaceutical carrier.

The present invention provides a method of making a modified brevinin-1 peptide composition for use as a medicament for the treatment of a bacterial infection comprising the steps of: providing a brevinin-1 peptide; modifying the brevinin-1 peptide to contain a —CONH₂ group to form a modified brevinin-1 peptide having at least 85% homology to SEQ ID NOS: 7-12. The present invention provides a cDNA composition encoding the modified brevinin-1 peptide disposed in a vector.

The modified brevinin-1 peptide may include a brevinin-1 HYba1 peptide having a sequence selected from SEQ ID NOS: 7-9, a brevinin-1 HYba2 peptide selected from SEQ ID NOS: 10-12 or both. The modified brevinin-1 peptide may have at least 85% homology to any sequence selected from SEQ ID NOS: 7-12. The modified brevinin-1 peptide may have at least 85% homology to SEQ ID NO: 7 or 10. The modified brevinin-1 peptide may have at least 85% homology to SEQ ID NO: 8 or 11. The modified brevinin-1 peptide may have at least 85% homology to SEQ ID NO: 9 or 12. The at least 85% homology may be 86, 87, 88, 89, 90, 91, 92, 93, 94, 95, 96, 97, 98, 99, 99.5, 99.8, or 100% homology. The pharmaceutical carrier may be a liposome, an ointment, a paste, a solution, a hydrogel, a gel, a petroleum carrier, a polymer, or a combination thereof.

The present invention provides an antimicrobial composition for the treatment of a bacterium, wherein the composition comprises, the modified brevinin-1 peptide comprises a brevinin-1 HYba1 peptide having a sequence selected from SEQ ID NOS: 7-9, a brevinin-1 HYba2 peptide selected from SEQ ID NOS: 10-12 or both. The first active agent may be amoxicillin, doxycycline, cephalexin, ciprofloxacin, clindamycin, metronidazole, azithromycin, sulfamethoxazole/trimethoprim, amoxicillin/clavulanate, levofloxacin, clotrimazole, econazole nitrate, miconazole, terbinafine, fluconazole, ketoconazole, or amphotericin.

### BRIEF DESCRIPTION OF THE DRAWINGS

For a more complete understanding of the features and advantages of the present invention, reference is now made to the detailed description of the invention along with the accompanying figures and in which:

FIG. 1 is an image of a helical wheel projection of both the peptides showing that they are amphipathic peptides, wherein the hydrophobic residues are aligned on one side of the helix.

FIGS. 2A-2F are graphs of the killing kinetics for S. aureus and V. cholerae evaluated to estimate the time taken to kill the microorganism at Minimum Inhibitory Concentration (MIC) concentration of the 4 peptides.

FIGS. 3A, 3B, 3C and 3D are plots of the effect of divalent cations on peptide-membrane interaction for S. aureus and V. cholerae.

FIGS. 4A and 4B are circular dichroism images which reveals an alpha helical structure of the peptides in the presence of TFE and SUVs, which mimics a membrane environment.

FIGS. 5A-5L (S. aureus) and FIGS. 6A-6K and 6M (V. cholera) are images of the bacterial membrane permeation by the amidated peptides.

FIGS. 7A-7F are images of FACS analysis of membrane depolarization induced by brevinin-1 HYba1 and 2 for S. aureus and V. cholerae.

FIGS. 8A-8N are images of the evaluation of peptide concentration-dependent bacterial membrane damage for S. aureus and V. cholerae.

FIGS. 9A-9J are scanning electron microscopy images visualizing the changes in surface morphology of bacteria for S. aureus and V. cholerae.

FIGS. 10A-10F are atomic force microscopy images visualizing the changes in surface morphology of bacteria for V. cholerae.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Frog skin secretion is a rich source of biologically active molecules and this is especially true of members of the endemic amphibian fauna of Western Ghats, India. Brevinins are a group of omnipresent peptides reported from many amphibian species. Two unique brevinin-1 peptides (brevinin-1 HYba1 and brevinin-1 HYba2) have been identified and structurally characterized from the cDNA library of the skin secretion of Hydrophylax bahuvistara, an endemic frog species of Western Ghats. The mature peptides contain 24 residues with a variable amino acid residue at the 10th position. Upon amidation, both the peptides showed increased activity and killing kinetics against gram-positive and gram-negative bacteria without altering their hemolytic property. Influence of cations (Mg²⁺ and Ca²⁺) on the peptide activity was found to be contrasting for gram-positive and negative bacteria. For elucidating the mechanism of action of these peptides on the bacteria tested, we employed flow cytometry using a voltage-sensitive dye which revealed that peptide-membrane interaction was primarily initiated by membrane depolarization. This is followed by pore formation without a change in cell morphology as per the Confocal Laser Scanning Microscopy and Scanning Electron Microscopy observations. The changes observed in bacterial cell structure at different time points of peptide interaction were documented. The ‘climax’ structures like bacterial aggregates and clumps under peptide challenge were observed by Scanning Electron Microscopy and confirmed by Atomic Force Microscopy. The present study highlights the potential of structural modifications that enhances the therapeutic potential of the brevinin-1 family of peptides.

The following abbreviations are used herein, HPLC: High Performance Liquid Chromatography; MALDI-TOF: Matrix-Assisted Laser Desorption Ionization Time of Flight; RACE: Rapid Amplification cDNA Ends; TFA: trifluoroacetic acid; MIC: Minimum Inhibitory Concentration; AMP: antimicrobial Peptide; HDP: Host Defense Peptides; CLSM: Confocal Laser Scanning Microscopy; SEM: Scanning Electron Microscopy; AFM: Atomic Force Microscopy; FACS: Fluorescence-activated cell sorting

Search for novel antimicrobial agents is attaining momentum because of the emergence of antimicrobial resistance mechanisms developed by microbes. Molecular diversity of skin active compounds derived from the endemic fauna is considered as a potential source for the development of new antimicrobial agents [1]. Among them, peptide based drugs are now receiving attention since they represent a viable and fertile area for drug discovery [2, 3]. Since the majority of the current drugs are based on natural products, integrated approaches for identifying novel molecules trapped in unexploited organisms with a view to combating human and animal disease have currently increased [4].

Host Defense Peptides (HDPs) are a part of the innate immune system, previously referred to as antimicrobial peptides, and have become the prime focus for drug development due to its peculiar mode of action. Being cationic, they are electrostatistically attracted towards anionic membranes and disrupt the integrity of the bacterial membrane. They can also travel across cytoplasmic membranes of the bacteria and obstruct vital metabolic processes [5]. The multiple modes of action employed by antimicrobial peptides are believed to reduce the ability of microorganisms to develop resistance against these peptides [6].

Even though HDPs are isolated from various sources, amphibian HDPs hold a special position because of their amphibious mode of life. Evolutionarily, they need protection from both land and water. Hence, their immune system is so evolved to face the challenges of both terrestrial and aquatic environments by developing HDPs in their skin secretion [7]. Lack of scales or body armor might have forced the evolutionary process to confer the immunity role on the skin [8, 9]. Immune function of the skin rests on the dermal glands that are found either in localized regions or randomly distributed on the dorsal surface. Cytoplasm of the gland cells are tightly packed with granules containing the peptides [10]. They are released in a holocrine manner upon contraction of the encircling myocytes [11]. Apart from the skin glands, HDPs are also produced from the mucosal lining of the respiratory and gastrointestinal tract [12]. HDPs produced by the glands inhibit the growth of microorganisms or upset predator physiology [13, 14].

HDPs from amphibians are grouped under the Frog Skin Active Peptide Family (FSAP family), which is again categorized on the basis of their biological function as (a) antimicrobial peptides (AMP) (b) smooth muscle active peptides (c) nervous system active peptides [15]. AMPs are potential candidates for the development of a novel group of antibiotics because their primary target is biological membranes and there are fewer chances to develop resistance against AMPs [16, 17]. The second and third category could act as agonist or antagonist of hormones/signaling molecules which reveal their pharmaceutical relevance [15]. Amphibian HDPs are gene derived and translated as a large peptide (prepropeptide) with an N-terminal signal sequence (pre-region), an acidic spacer (pro-region) that terminates in a dibasic cleavage site (e.g. KR, KK) [18] followed by a C-terminal mature peptide. These peptides are synthesized through the secretary pathway, the signal sequence targets the peptide to the endoplasmic reticulum, the spacer is cleaved to release mature peptide by trypsin-like enzymes at the time of secretion. These are usually cationic, to target anionic membranes and α-helical with 40-50% hydrophobic residues that cluster on one face when they attain helical structure in a hydrophobic environment, they are unstructured in aqueous solution [19, 20]. Apart from the general antimicrobial effect, HDPS have diverse functions, which include anticancer effect [21, 22], immune system activation [23], antiviral [24] and anti-fungal activities [25]. It was hypothesized recently that AMPs in frog skin act like a cytolysine which assisted the delivery of neuroactive peptides to the endocrine and nervous system of the predator [7]. Various methods such as sequence modification, minimalist approach, combinatorial libraries and template assisted approach have been proposed for designing new antimicrobial peptides. Among them sequence modification by deleting, adding, replacing residues or by truncating the N and C terminals of the peptide is the most preferred approach and C-terminal amidation is the most studied post-translational modification as it is commonly seen in natural peptides [26]. Recent reports revealed an enhancement of antimicrobial activity of peptides upon C-terminal amidation [27].

Until the present invention, a handful of frog species from limited geographical locations have been studied for skin HDPs. The hidden diversity of HDPs in Asian frogs and the current status of HDP research in Asia have been reviewed recently [28]. The Western Ghats of India is considered as a mega biodiverse area with high level of amphibian endemism. Since the hypothesis that two frog species never have the peptides with the same sequence structure [17], this endemic fauna offers a unique model system to explore the hitherto unexplored novel molecules present in their skin secretions. Among the 223 amphibian species distributed in the Western Ghats, only 4 species [29-34] have been explored for skin active peptides and there is, thus, a need to develop a skin peptide library of other endemic frogs of this region. Hydrophylax bahuvistara [35] commonly known as Fungoid frog, is an endemic frog species of the Western Ghats of India.

As a part of the search for novel HDPs, the present study attempts to assess the antimicrobial activity and mode of action of peptides from the skin secretion of Hydrophylax bahuvistara. Here, we report the identification and characterization of two novel brevinin-1 peptides and their structural analogs, utilizing shotgun cloning followed by chemical synthesis. Bioassays were performed and mode of action of these peptides was studied further.

Skin secretion harvesting: Adult specimens of H. bahuvistara (both sexes, n=5) were collected from the Northern part (Kanhangad) of Kerala, under the license from Kerala Forest Department, India. Skin secretion from each specimen was collected by giving a mild transdermal electrical stimulation (6 v DC, 4 ms pulse width, 50 HZ) for 20 s duration [36]. During the electrical stimulation, the skin was rinsed with Milli-Q H₂O and the aqueous solution was collected and immediately fixed in liquid nitrogen, brought back to the laboratory, lyophilized and stored at −80° C. prior to analysis. The frogs were released in a healthy state back to the same habitatit was collected from. No adverse events were noticed in the specimens after stimulation.

Molecular cloning of cDNAs encoding antimicrobial peptides: Poly (A) mRNAs were isolated from the lyophilized secretion using DYNA BEADS® (Dynal Biotech, UK) in accordance with manufacturer's instructions. cDNA library was constructed using SMARTer™cDNA Amplification Kit (Clontech, UK) in agreement with manufacturer's instructions. SMART MMLV RT and the primers, SMARTer II A, Oligonucleotide Primer SEQ ID NO: 1 5′-AAGCAGTGGTATCAACGCAGAGTACGCGGG-3′ and 3′CDS Primer A SEQ ID NO: 2 5′-AAGCAGTGGTATCAACGCAGAGTAC (T) 30VN-3′ (N=A, C, G or T; V=A, G, C) were used to synthesize the first-strand cDNA. Advantage DNA Polymerase was used to amplify the second strand by the primers 3′CDS Primer A and 5′ PCR primer SEQ ID NO: 3 5′-AAGCAGTGGTATCAACGCAGAGT-3′. Screening of cDNAs encoding antimicrobial peptides was carried out with two sense primers, including a specific primer designed for ranid frogs from the nucleotide sequence of the highly conserved signal peptide region and 5′-untranslated region of antimicrobial peptide-encoding cDNAs and a degenerate primer (SEQ ID NO: 4 5′-GAWYYAYY HRAGCCYAAADATG-3′). 3′CDS primer A was used as the anti-sense primer. Advantage DNA Polymerase (Clontech, UK) was used for PCR with the following conditions: 94° C. for 2 min; followed by 30 cycles of 92° C. for 10 s, 50° C. for 30 s, 72° C. for 40 s; and again followed by a final extension at 72° C. for 10 min. Gel purified PCR products were cloned into pGEM-T easy vector system (Promega Corp.) followed by plasmid isolation. Purified plasmids were sequenced using ABI 3730 automated sequencer. Nucleotide sequences obtained were translated using EMBOSS transeq. The peptide sequence obtained was subjected to homology searches using BLAST (NCBI) to confirm their identity. Among them two novel peptides belonging to brevinin-1 family (brevinin-1 HYba1 and brevinin-1 HYba2) were selected for further studies.

Physico-chemical properties of the Host Defense Peptides: Net charge and grand average of hydropathicity (GRAVY) of the peptides were computed using ProtParam. Peptide Synthetics were used to calculate the theoretical molecular mass of the peptides. Helical wheel of the peptides was plotted to predict their functional roles using Don Armstrong and Raphael Zidovetzki (Version: Id: wheel.pl, v1.42009-10-2021:23:36don Exp). Secondary structure prediction from amino acid sequences were performed using PSIPRED and jpred4 prediction methods [37, 38].

Design and synthesis of brevinin-1 peptides and their analogs. Both brevinin-1 HYba1 (B1/1) and brevinin-1 HYba2 (B1/2) were synthesized in 3 forms, with C-terminal acid/natural (B1/1 COOH and B1/2 COOH), C-terminal amide (B1/1 CONH₂ and B1/2 CONH₂) and C-terminal amide and disulfide linkage (cyclic B1/1 CONH₂ and cyclic B1/2 CONH₂). C-terminal amidated peptides were synthesized by the stepwise manual 9-fluorenylmethoxycarbonyl (F_{moc}) solid phase peptide synthesis technique using CLEAR™ amide resin. Following deprotection and cleavage from the resin, the peptides were purified by reverse-phase HPLC. The purity of the final products was checked by MALDI-TOF MS. C-terminal acidic and cyclic amidated peptides were purchased from Synpeptide, Shanghai, China and the purity of the final products were checked with MALDI-TOF MS.

Antimicrobial activity: Broth dilution method [39] was used to assess the antimicrobial activity of the peptides. Bacterial strains used for in vitro antibacterial assay were Staphylococcus aureus (MTCC 9542), Bacillus subtilis (MTCC 14416), Bacillus coagulans (ATCC 7050), Methicillin-resistant Staphylococcus aureus (MRSA) (ATCC 43300), Streptococcus mutans (MTCC 497), Streptococcus gordonii (MTCC 2695), Vibrio cholerae (MCV09), Escherichia coli (ATCC 25922), Vancomycin-resistant enterococcus (VRE) (ATCC 29212) and gram-negative fish pathogens Aeromonas hydrophila (ATCC 7966) and Aeromonas sobria (ATCC 43979). Bacterial cultures were grown in Muller Hinton broth (MHB) (Hi-media) by overnight incubation at 37° C. with constant shaking. Microbial cultures having 10⁶ CFU/ml were made from OD₆₀₀: 0.6 cultures. 400 μM stock solutions of peptides were prepared in autoclaved double distilled water and diluted in MHB to make concentrations ranging from 0.7 to 100 μM. Bacterial inoculum without peptide was used as the negative control. The minimum inhibitory concentration (MIC) was taken as the minimum peptide concentration which exhibited 100% bacterial killing in 24 hrs. The assay was repeated thrice and the mean MICs of each microorganism used were compared between the three groups using two tailed student's t-test.

Killing kinetics. Killing kinetic analysis of the B1/1 CONH₂ and B1/2 CONH₂ and B1/1 COOH and B1/2 COOH against Gram-negative V. cholerae MCV09 and Gram-positive S. aureus (MTCC 9542) were carried out at its MIC and sub-MIC concentrations. Cells in mid-logarithmic growth phase were diluted to get 10⁶ CFU/ml (OD₆₀₀: 0.06) and incubated with the peptides in multiple micro titer wells. Aliquots were drawn at different time points for 24 hours and plated on MH agar. The number of colonies was counted after incubating the plates at 37° C. for 24 hrs. Cells without peptide treatment were taken as the positive control.

Hemolytic Activity: Hemolytic assay was carried out as previously described [40]. Briefly, 10% (v/v) suspensions of fresh human erythrocytes in phosphate buffered saline (pH 7.2) were incubated with different concentrations (100 μM-0.7 μM) of B1/1 COOH & B1/2 COOH, B1/1 CON_{H2} & B1/2 CON_{H2} and cyclic B1/1 CON_{H2} & cyclic B1/2 CON_{H2} and incubated at 3⁷° C. for 60 min. The cells were centrifuged (3000×g) for 5 min, and absorbance of the supernatant was measured at 595 nm. Hemolysis caused by 10% Triton X-100 was taken as positive control. Percentage hemolysis was calculated by measuring the mean amount of hemoglobin released as a result of lysis of erythrocyte from three independent experiments.

Effect of divalent cations on peptide-membrane interaction. The competing ability of B1/1 CONH₂ and B1/2 CONH2 for divalent cation binding sites on bacterial membranes were tested by determining MICs of the peptides in the presence of 20 mM Mg²⁺ and Ca²⁺[41,42]. V. cholerae (MCV09) and S. aureus (MTCC 9542) were prepared as above and incubated with different concentrations of peptides at 37° C. for 24 hrs. MHB used in the assay was altered by the addition of MgCl₂ and CaCl₂. Controls were used without the ions. Mean MICs were compared with the controls using two tailed student's t-test.

Preparation of small unilamellar vesicles (SUVs) for structural analysis of peptides using CD spectroscopy. The tendency of the B1/1 CONH₂ and B1/2 CONH₂ peptides to assume secondary structure in hydrophobic environments was investigated using spectropolarimeter (Jasco, Tokyo, Japan). Amidated brevinin-1 peptides (250 μg) were taken in three different media: sodium phosphate buffer (10 mM, pH: 7.4), trifluoroethanol (TFE)-water (30%, v/v) and small unilamellar vesicles (SUV) composed of 2-oleoyl-1-palmitoyl-sn-glycero-3-Phosphocholine (POPC), 1-palmitoyl-2-oleoyl-sn-glycero-3-phosphoglycerol (POPG): equimolar mixture (50:50). Each of these solutions was taken in a 1 mm path length quartz cuvette, scanned from 190 to 250 nm at 25° C. with a band width of 1 nm with scanning speed of 50 nm/min. The solvent CD signal was subtracted from the mean spectrum of three consecutive scans. The mean residue ellipticity was plotted against wavelength.

Imaging the bacterial membrane permeation by the amidated peptides. In order to find out the membrane damage by the peptides, if any, we used SYTOX green uptake assay. MIC concentrations of B1/1 CONH₂ and B1/2 CONH₂ were prepared in sodium phosphate buffer (10 mM, pH: 7.4). Overnight cultures of S. aureus (MTCC 9542) and V. cholerae (MCV09) were re-inoculated in fresh MHB to attain an OD₆₀₀-0.6. The bacterial suspension was centrifuged (3000×g) for 5 min and the pellet was washed twice with the sodium phosphate buffer (10 mM, pH: 7.4). The pellet was resuspended in the same buffer to an OD₆₀₀-0.06. Diluted culture was incubated with the peptides for 10 min at 37° C. 100 μl suspensions were poured on to poly-L-lysine coated glass slides and incubated at 37° c. for 30 minutes. The glass slides were washed twice in the same buffer to remove unattached cells. After washing 50 μl of DAPI (10 μg/ml) was smeared on the glass slide and incubated for 30 minutes at 37° C. The slides were washed twice with the buffer and SYTOX green (0.1 μM) was smeared and incubated for 15 minutes at room temperature, washed twice and dried. A drop of glycerol was placed on the slides, mounted with a cover slip and sealed. Controls were run in the presence of peptide solvents. The slides were subjected to confocal laser scanning microscopy (CLSM).

Evaluation of bacterial membrane depolarization induced by the peptides. S. aureus (MTCC 9542) and V. cholerae (MCV09) cells were incubated with the B1/1 CONH₂ and B1/2 CONH₂ at their respective MICs for 10 min at 37° C. and the membrane potential sensitive dye bis-(1,3-dibutylbarbituric acid) trimethine oxanol [DiBAC4 (3)] (1 μg/ml) was added to it. The cell suspension was centrifuged (3000×g) for 5 min and the pellets obtained were suspended in 500 μl sodium phosphate buffer (10 mM, pH 7.4). Depolarization induced by the peptides was measured using flow cytometer at an excitation wavelength of 490 nm and the emission maximum at 516 nm [43]. The green fluorescence in the channel FL1 was measured. For each sample 10,000-30,000 events were analyzed. DIVA software (BD) was used for data acquisition and analysis. The Forward Scatter Side Scatter Dot Plot referring to relative cell size, granularity of bacterial population was differentiated from the background signals and gated for evaluation of the fluorescence. To gate the viable cells in the control, a marker was plotted.

Evaluation of Peptide Concentration-dependent bacterial membrane damage as previously described with modifications [44]. S aureus (MTCC 9542) and V. cholerae (MCV09) were grown in MHB at 37° C., washed, and suspended in sodium phosphate buffer (10 mM, pH: 7.4) (OD 600-0.6). Diluted bacteria were incubated with the B1/1 CONH₂ and B1/2 CONH₂ at 3 different concentrations (S aureus: Sub-MIC; 0.7 μM for both peptides, MIC; table 3 and supra-MIC; 5 μM for both peptides, V. cholerae: Sub-MIC; 5 μM for both peptides, MIC; table 3 and supra-MIC; 25 μM for both peptides) for 10 min at 37° C. 0.1 μM SYTOX green was added and incubated and the increase in fluorescence was monitored in a flow cytometer (excitation wavelength of 485 nm and emission wavelength of 520 nm) using the settings described above.

Visualizing the changes in surface morphology of bacteria: Scanning Electron Microscopy. For documenting the changes that occur on bacteria under B1/1 CONH₂ and B1/2 CONH₂ challenge, SEM experiments were performed. The overnight culture of S. aureus (MTCC 9542) and V. cholerae (MCV09) in MHB were re-inoculated and incubated for 3-4 hours to get mid-log phase cells (OD₆₀₀-0.6). These cells were centrifuged (3000×g) for 5 min and the pellet was washed twice in sodium phosphate buffer (10 mM, pH: 7.4) and diluted to an OD₆₀₀ value 0.1. B1/1 CONH₂ and B1/2 CONH₂ was diluted to MIC concentration with the same buffer. Peptides were added to the diluted culture and incubated at 37° C. Samples were taken at two-time points (10 min and 15 min) of incubation. After incubation the peptide-bacteria suspension was centrifuged at (3000×g) for 5 min, the pellet was washed twice with sodium phosphate buffer (10 mM, pH: 7.4). Subsequently, the bacterial pellet was chemically prefixed with 500 μl 2.5% glutaraldehyde (v/v) for 1 hour at 4° C. The pellet was washed twice with the buffer and subsequently dehydrated with graded acetone series (30%, 50%, 70%, 90%, 100%, 100%, and 100%) for 15 min each. The pellet was dried in vacuum desiccator. The dried sample was analyzed with scanning electron microscope (Jeol, USA).

Visualizing the changes in surface morphology of bacteria: Atomic Force Microscopy. This was incorporated to get more insights into the mechanism of action of the peptides and to confirm the results obtained from SEM. V. cholerae cells (MCV09) were grown in MHB at 37° C., washed, and suspended in sodium phosphate buffer (10 mM, pH: 7.4) (OD₆₀₀-0.06). Diluted bacteria were incubated with the MICs of B1/1 CONH₂ and B1/2 CONH₂ for 15 min. The control sample was prepared without the peptides. Samples were prepared by drop casting 20 μL of a solution on the freshly cleaved mica surface and dried under air. AFM analyses were carried out on Multimode SPM (Veeco Nanoscope V). Imaging was done under ambient conditions in tapping mode. The probe used for imaging was antimony doped silicon cantilever with a resonant frequency of 300 kHz and a spring constant of 40 Nm⁻¹.

Molecular cloning of cDNAs encoding HDPs. Two cDNA sequences, encoding brevinin-1 were obtained from the skin cDNA library of H. bahuvistara. The nucleic acid sequences of each cDNA were confirmed in at least five replicates. Table 1 illustrates the deduced amino acid sequences of the two peptides.

TABLE 1
Open Reading Frame Amino Acid Sequences of brevinin-1 Hyba peptides

| SEQ | | | |
|---|---|---|---|
| ID | | | |
| NO: | Putative Signal Sequence | Acidic Spacer | Mature Peptide |
| 5 | MFTLKKCMLLIFFLGTINESLC | QEESNAEEERRDDDDDQMNVEVEKR | FFPGIIKVASAILPTAICAITKRC |
| 6 | MFTLKKPLELIFFLGTINESLC | QEESNAEEERRDDDDDQMNVEVEKR | FFPGIIKVAGAILPTAICAITKRC |

SEQ ID NO: 5 was named brevinin-1 HYbaQ while SEQ ID NO: 6 was named brevinin-1 HYba2. *dibasic cleavage site of acidic spacer and the 10th position amino acid of the mature peptide are highlighted.

The peptides differed in the 10th amino acid, hence considered as paralogs. NCBI BLAST search revealed that both the peptides showed 67% similarity with brevinin-1 SN1 from Sylvirana spinulosa [45]. They also possess a Rana Box and conserved amino acid residues—characteristic feature of Brevinin-1 family of peptides. The peptides were named as brevinin-HYba1 and brevinin-1 HYba2 respectively according to the proposed nomenclature system [46] for frog skin peptides. Open reading frame encoding the peptide precursors of both the paralogs consisted of 71 amino acid residues. The mature peptides contain 24 residues (Table 1). The conserved pre pro-regions of each precursor open reading frame contain a putative signal peptide of 22 amino acids followed by an acidic spacer that terminates in a dibasic cleavage site Lys-Arg (K-R) (Table 1).

Physicochemical Properties of the Host Defense Peptides brevinin-1 HYba1 and brevinin 1 HYba2. It was found that both the peptides are cationic with net charge +3 at pH 7, hydrophobicity 62% and a GRAVY value of 1.2 (Table 2a/b). Cationic peptides with more than 50% hydrophobicity usually tend to be potent antimicrobial agents.

TABLE 2a
Physico-chemical Properties of the peptides

| | SEQ | | No: | Secondary Structure | |
|---|---|---|---|---|---|
| | ID | | of | Prediction | |
| Peptide Name | NO: | Peptide Sequence | Residues | JRED 4 | PSIPRED |
| brevinin-1 | | | | | |
| HYba 1 | | | | | |
| B1/1 COOH | 7 | FFPGIIKVASAILPTAICAITKRC | 24 | α helix | α helix |
| | | | | (I5-T21) | (P3-R23) |
| B1/1 CONH₂ | 8 | FFPGIIKVASAILPTAICAITKRC- | 24 | | |
| | | NH2 | | | |
| Cyclic B1/1 | 9 | FFPGIIKVASAILPTAICAITKR | 24 | | |
| CONH₂ (C18- | | C-NH2 | | | |
| C24) | | | | | |
| brevinin-1 | | | | | |
| HYba2 | | | | | |
| B1/2 COOH | 10 | FFPGIIKVAGAILPTAICAITKRC | 24 | α helix | α helix |
| | | | | (I5-T21) | (G4-K22) |
| B1/2 CONH₂ | 11 | FFPGIIKVAGAILPTAICAITKRC- | 24 | | |
| | | NH2 | | | |
| Cyclic B1/2 | 12 | FFPGIIKVAGAILPTAICAI | 24 | | |
| CONH₂ (C18- | | TKRC-NH2 | | | |
| C24) | | | | | |

TABLE 2b
Physico-chemical Properties of the peptides

| | | | | | | Hydro- | |
|---|---|---|---|---|---|---|---|
| | SEQ | | | Ex- | Observed | phobi- | |
| | ID | | Net | pected | Mass | city | |
| Peptide Name | NO: | Peptide Sequence | Charge | Mass | [M + 3H]3+ | (%) | GRAVY |
| brevinin-1 | | | | | | | |
| HYba 1 | | | | | | | |
| B1/1 COOH | 7 | FFPGIIKVASAILPTAICAIT | 3 | 2534.1 | 2537 | 62% | 1.258 |
| | | KRC | | | | | |
| B1/1 CONH₂ | 8 | FFPGIIKVASAILPTAICAIT | 4 | 2533.1 | 2536.1 | | |
| | | KRC-NH2 | | | | | |
| Cyclic B1/1 | 9 | FFPGIIKVASAILPTAICAITK | 4 | 2531.1 | 2534.1 | | |
| CONH₂ (C18- | | C-NH2 | | | | | |
| C24) | | | | | | | |
| brevinin-1 | | | | | | | |
| HYba2 | | | | | | | |
| B1/2 COOH | 10 | FFPGIIKVAGAILPTAICAIT | 3 | 2504.19 | 2507 | 62% | 1.275 |
| | | KRC | | | | | |
| B1/2 CONH₂ | 11 | FFPGIIKVAGAILPTAICAIT | 4 | 2503.1 | 2506.2 | | |
| | | KRC-NH2 | | | | | |
| Cyclic B1/2 | 12 | FFPGIIKVAGAILPTAICAI | 4 | 2501.1 | 2504.1 | | |
| CONH₂ (C18- | | TKRC-NH2 | | | | | |
| C24) | | | | | | | |

A positive value of GRAVY for both the peptides adds to their antimicrobial property. Another feature that is required by a candidate peptide is its helical structure. Both the sequences were predicted to be helical by PSIPRED and Jpred 4 methods (Table 2a).

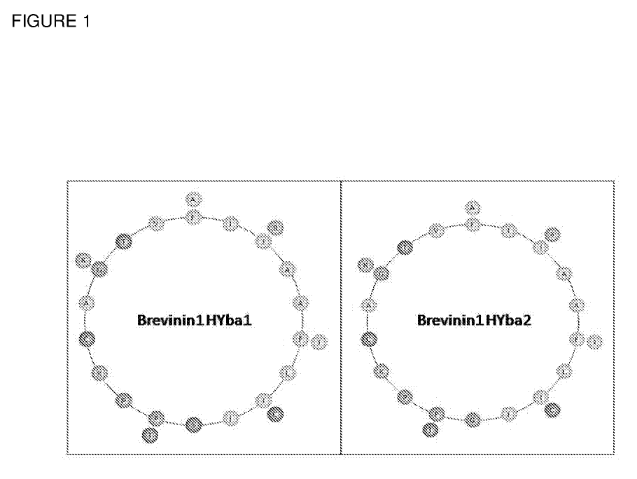

FIG. 1 is an image of a helical wheel projection of both the peptides showed that they are amphipathic peptides, wherein the hydrophobic residues are aligned on one side of the helix. Designing of analogs and Solid Phase Peptide Synthesis (SPSS). Six peptides (B1/1 COOH (SEQ ID NO: 7), B1/2 COOH (SEQ ID NO: 8), B1/1 CONH₂ (SEQ ID NO: 9), B1/2 CONH₂ (SEQ ID NO: 10), cyclic B1/1 CONH₂ (SEQ ID NO: 11) and (SEQ ID NO: 12) cyclic B1/2 CONH₂) were synthesized and their purity and mass were confirmed using HPLC and MALDI TOF MS.

TABLE 3
Comparison of Antimicrobial activity of natural brevinin-1 peptides
and their synthetic Analogs

| | | | Cyclic | | | Cyclic |
|---|---|---|---|---|---|---|
| | B1/1 | B1/1 | B1/1 | B1/2 | B1/2 | B1/2 |
| | COOH | CONH₂ | CONH₂ | COOH | CONH₂ | CONH₂ |
| Gram-positive | | | | | | |
| (MICμM)* | | | | | | |
| Staphylococcus | 9.5 | 1.5 | 3 | 12.5 | 2.5 | 3 |
| aureus | | | | | | |
| MTCC 9542 | | | | | | |
| Bacillus subtilis | 98 | 36.3 | 45 | 67.5 | 30 | 35 |
| MTCC 14416 | | | | | | |
| Bacillus | 25 | 8.2 | 25 | 25 | 12.5 | 29 |
| coagulans | | | | | | |
| ATCC 7050 | | | | | | |
| MRSA ATCC | 25 | 2.5 | 5 | 30 | 5 | 7 |
| 43300 | | | | | | |
| VRE ATCC | 50 | 25 | 25 | 50 | 25 | 30 |
| 29212 | | | | | | |
| Streptococcus | NA | 36 | 40 | NA | 40 | 40 |
| mutans MTCC | | | | | | |
| 497 | | | | | | |
| Streptococcus | NA | 19 | 25 | NA | 25 | 25 |
| gordonii | | | | | | |
| MTCC 2695 | | | | | | |
| Gram-negative | | | | | | |
| Vibrio cholerae | NA | 10.3 | 12.5 | NA | 12 | 12.5 |
| MCV09 | | | | | | |
| E .coli. ATCC | NA | 29 | 50 | NA | 36.2 | 50 |
| 25922 | | | | | | |
| Fish pathogens | | | | | | |
| (Gram-negative ) | | | | | | |
| Aeromonas | NA | 100 | 100 | NA | 100 | 50 |
| hydrophilia | | | | | | |
| ATCC 7966 | | | | | | |
| Aeromonas | NA | 12.5 | 3 | NA | 12.5 | 3 |
| sobria | | | | | | |
| ATCC 43979 | | | | | | |

*MIC represents the lowest peptide concentration required to kill entire bacteria,
NA-not active up to the highest concentration tested.

TABLE 4
Percentage hemolysis at MIC of brevinin-1 Peptides and
their Structural Analogs

| | | | | | |
|---|---|---|---|---|---|
| | | Cyclic | | | Cyclic |
| B1/1 | B1/1 | B1/1 | B1/2 | B1/2 | B1/2 |
| COOH | CONH₂ | CONH₂ | COOH | CONH₂ | CONH₂ |

Gram-positive

| | | | | | | |
|---|---|---|---|---|---|---|
| Staphylococcus | 20 (9.5) | 10(1.5) | 11 (3) | 25(12.5) | 8(2.5) | 15(3) |
| aureus | | | | | | |
| MTCC 9542 | | | | | | |

Gram-negative

| | | | | | | |
|---|---|---|---|---|---|---|
| Vibrio cholerae | NA | 20(10.3) | 20(12.5) | NA | 25(12) | 25(12.5) |
| MCV09 | | | | | | |

Fish pathogens (Gram-negative )

| | | | | | | |
|---|---|---|---|---|---|---|
| Aeromonas | NA | 25(12.5) | 10(3) | NA | 25(12.5) | 12(3) |
| sobria | | | | | | |
| ATCC 43979 | | | | | | |

*(MIC in μM is given in parenthesis)

Antimicrobial activity, killing kinetics and hemolysis. Table 3 demonstrates the MIC of the peptides evaluated against gram-positive and gram-negative bacteria. Natural brevinin-1 peptides (B1/1 COOH, B1/2 COOH) showed activity against some of the tested gram-positive bacteria S. aureus, B. subtilis, B. coagulans and MRSA with MICs ranging from 9 to 100 μM for B1/1 COOH and 9 to 70 μM for B1/2 COOH. These peptides were not active against gram-positive S. nutans and S. gordonii and all the other gram-negative bacteria including the fish pathogens tested. The MIC profile of amidated brevinin peptides against all the tested gram-positive bacteria ranged from 1 to 40 μM for B1/1 CONH₂ and 2.8 to 40 μM for B1/2 CONH₂. C-terminal amidation gained activity against gram-negative bacteria in a range of 10-50 μM for both B1/1 CONH₂ and B1/2 CONH₂. Both the amidated peptides were active against fish pathogens (12-100 μM). MICs of C-terminal amidated cyclic peptides were more or less the same as B1/1 CONH₂ and B1/2 CONH₂, except for A. sobria (Table 3). Two-tailed student's t test was done to determine whether the difference in MIC exhibited by the peptide due to modifications was significant or not. Statistically significant difference in MICs values was obtained for S. aureus, MRSA, and A. sobria. In the case of S. aureus and MRSA reduction of MICs between B1/1 CONH₂ and B1/2 CONH₂ and B1/1 COOH and B1/2 COOH was significant (p<0.01). The reduction in MICs of A. sobria was significantly different between B1/1 CONH₂ and B1/2 CONH₂ and cyclic B1/1 CONH₂ and cyclic B12 CONH₂ (p<0.01). Considering the hemolytic activity (Table 4) of the peptides, these modifications retain their hemolytic nature. It does not show significant increase or decrease.

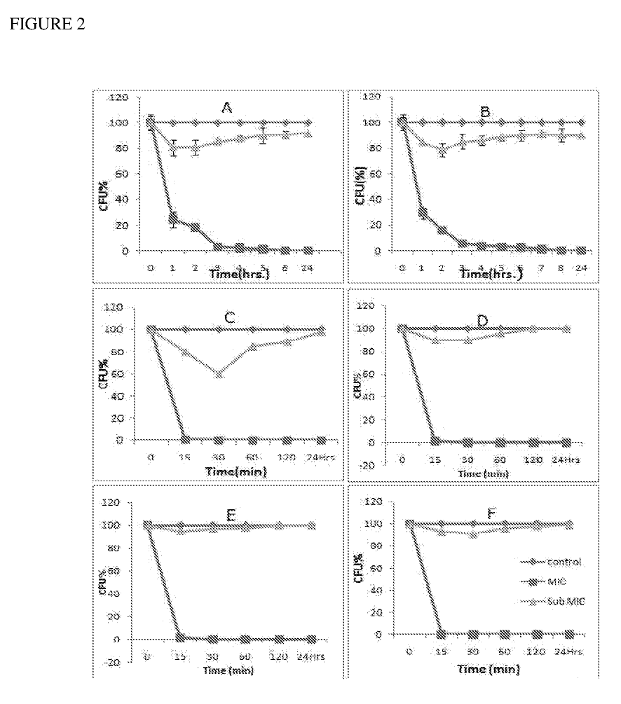

FIGS. 2A-2D are graphs of the killing kinetics for S. aureus and V. cholerae was evaluated to estimate the time taken to kill the microorganism at MIC concentration of the 4 peptides. Peptides with combinatorial modification were not assessed because they exhibited MIC more or less the same as that of amidated peptides. Sub-MIC concentration of the peptides was also plotted to demonstrate that their growth curve resembles that of negative control. Both the acidic peptides (B1/1 COOH and B1/2 COOH) took about 5-6 hours to completely eliminate the S. aureus. On amidation, the time taken was reduced to about 15 min. This reveals the role of PTMs influencing the activity of the peptides. Such a comparison was not possible for V. cholerae because only amidated forms were active and they eliminate the bacteria in about 15 minutes. Results of MIC and killing kinetics revealed that amidated peptides are more potent among the tested modifications. Hence, only the amidated analogs will be evaluated in the rest of the assays.

FIGS. 3A-3D are plots of the effect of divalent cations on peptide-membrane interaction. FIGS. 3A-3B show the effect of Ca²⁺ and Mg²⁺ ions on the activity of B1 HYba1 and B1 HYba2 against S. aureus. FIGS. 3C-3D show the effect of Ca²⁺ and Mg²⁺ ions on the activity of B1 HYba1 and B1 HYba2 against V. cholera. This was done in order to access whether the activity of the peptides are influenced by divalent cations (Mg²⁺ and Ca²⁺). The addition of the amidated peptides and (20 mM) Mg²⁺/Ca²⁺ to a culture of V. cholerae resulted in the complete abortion of antimicrobial activity (MIC>100 μM) (FIGS. 3C and 3D). Two-tailed student's t-test revealed that the difference was significant (p<0.01). The addition of the peptides and (20 mM) Mg²⁺/Ca²⁺ to a culture of S. aureus affected the antimicrobial activity, but still retained the ability to inhibit bacterial growth (5-12 μM) (FIGS. 3A and 3B). This shows that there is not much influence of salts on gram-positive bacterial membrane permeation under study.

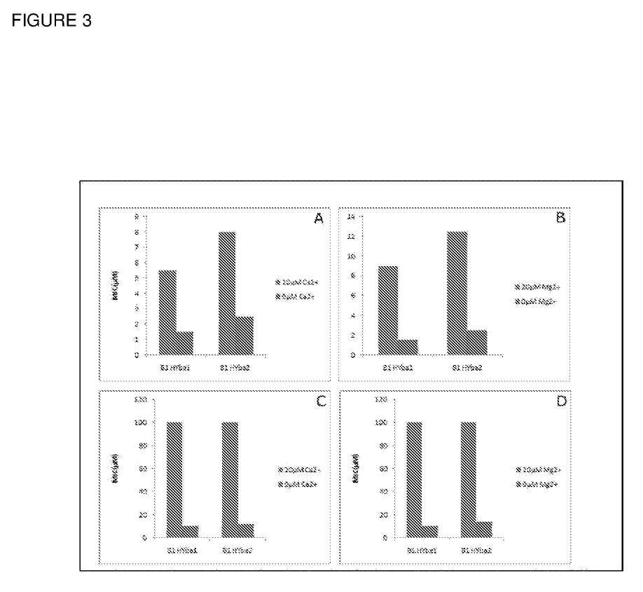

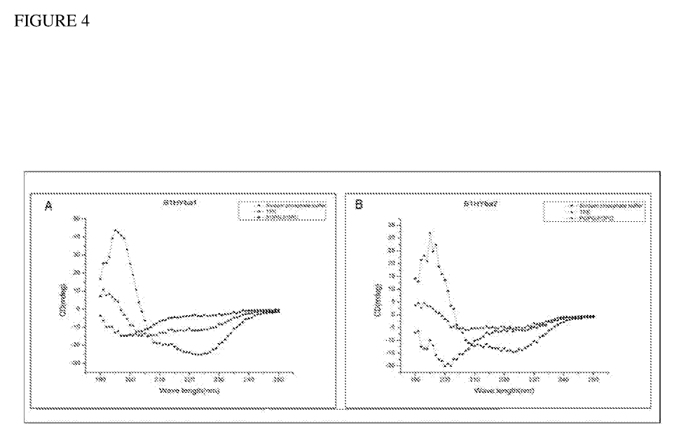

FIGS. 4A and 4B are circular dichroism image: The CD spectroscopy based secondary structural analysis showed that these peptides have a high propensity to adopt the alpha-helical conformation in membrane mimetic environment like TFE in water (FIG. 4). Both the amidated peptides attained a well-defined alpha-helical structure in anionic and bacterial membrane mimicking lipid environments (POPC/POPG) as indicated by a negative ellipticity and double minima at 208 and 222 nm.

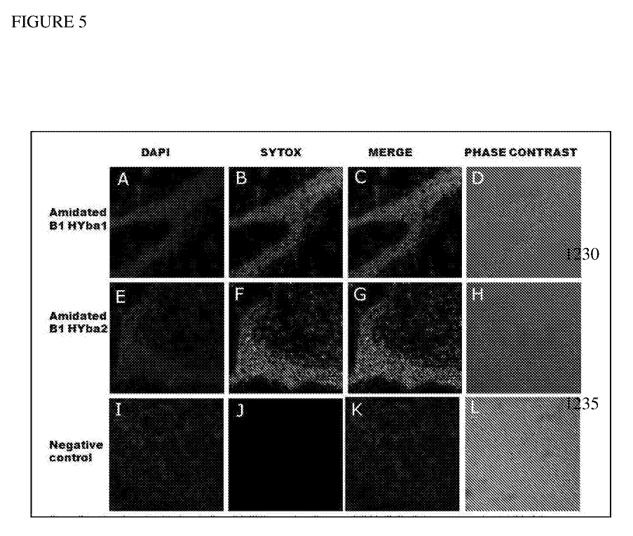

FIGS. 5A-5L (S. aureus) and FIGS. 6A-6K and 6M (V. cholera) are images of the bacterial membrane permeation by the amidated peptides. FIGS. 5A, 5E, and 5I show DAPI signal where all the bacterial cells could be visualized. FIGS. 5B, 5F and 5J show SYTOX signal, only membrane damaged cells emit the green signal (FIGS. 5B, 5F). FIGS. 5C, 5G and 5K show merged images combinatorial signals of DAPI and SYTOX. FIGS. 5D, 5H and 5L represent phase contrast images. FIGS. 6A, 6E, and 6I show DAPI signal where all the bacterial cells could be visualized. FIGS. 6B, 6F and 6J show SYTOX signal, only membrane damaged cells emit the green signal (FIGS. 6B, 6F). FIGS. 6C, 6G and 6K show merged images combinatorial signals of DAPI and SYTOX. FIGS. 6D, 6H and 6L represent phase contrast images. Double staining was used to visualize the total number of bacterial cells in the preparation and the cells that have undergone membrane permeabilization. As killing kinetics revealed 100% cell death at 15 minutes, incubation time was fixed to 10 minutes so as to observe changes occurring in intact cells. DAPI, the double strand binding blue fluorescent dye was used to stain all bacterial cells irrespective of membrane damage. SYTOX green, the green fluorescent DNA binding probe does not penetrate the bacterial membrane unless permeabilized by the peptide. A marked increase in fluorescence signal was observed in S. aureus and V. cholerae cells that were treated with MIC concentrations of B1/1 CONH₂ and B1/2 CONH₂ (FIGS. 5A-5L and FIGS. 6A-6K and 6M). There was no SYTOX green fluorescence from untreated cells. In FIGS. 5A-5L and FIGS. 6A-6K and 6M, the first panel shows DAPI signal where all the cells in the area could be visualized. The second panel is that of the cells affected by the peptide which emitted the SYTOX green signal. The third panel shows the merged image. As both the dyes used are DNA binding, a combinatorial signal (bluish green) can be observed in the merged images. These results confirm the membrane permeabilization of all the bacterial cells by both the peptides. The results also suggest that the primary targets of these peptides are bacterial membranes and they may have a membranolytic mechanism of action.

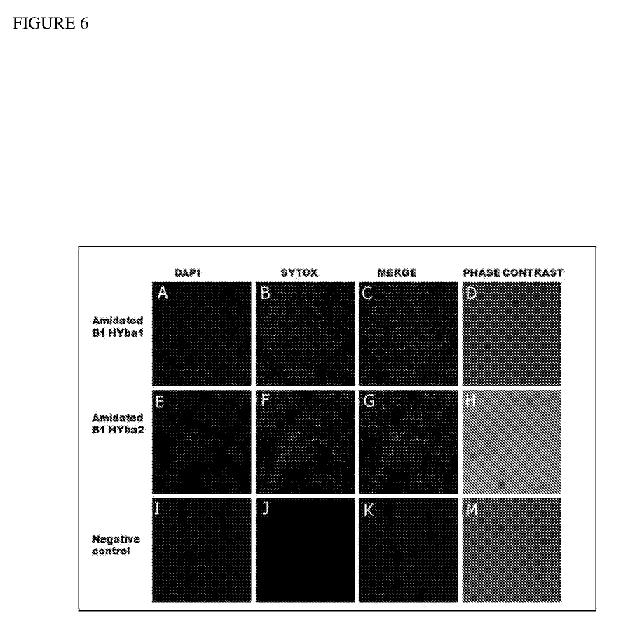

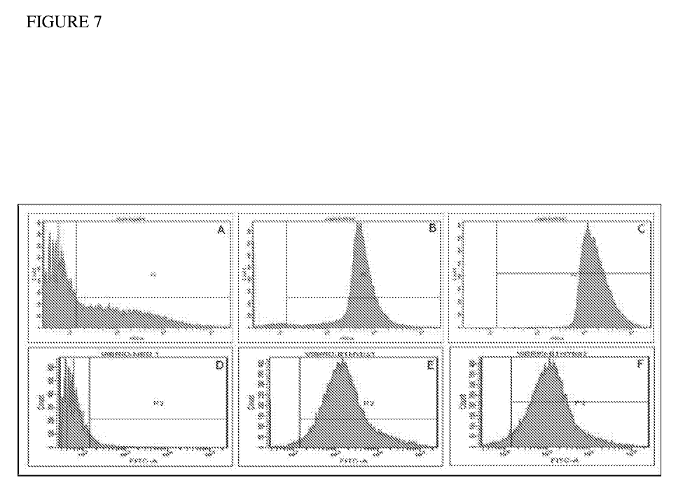

FIGS. 7A-7F are images of FACS analysis of membrane depolarization induced by brevinin-1 HYba 1 and 2. FIG. 7A shows untreated S. aureus cells; FIGS. 7B-C show S. aureus treated with MIC of B1 HYba1 & 2. FIG. 7D show untreated V. cholerae cells; FIGS. 7E-7F show peptide treated V. cholerae cells. Membrane depolarization is indicated by a shift in the population. Flow cytometric analysis revealed that both the amidated brevinin1 peptides at their MICs could depolarize the membranes of S. aureus and V. cholerae. This was indicated by a marked shift in the fluorescence peak of the voltage sensitive fluorescent dye DiBAC4 to the right from the negative control. Evaluation of bacterial membrane depolarization induced by the peptides. The ability of the peptides under study to depolarize the membrane of S. aureus and V. cholerae was investigated using a voltage sensitive fluorescent dye DiBAC4 (3). The dye binds to the bacterial membrane only when it is depolarized. Depolarization increases the permeability of DiBAC4 (3) and enables it to bind to intracellular lipids and proteins increasing its fluorescent signal, which is analyzed flow cytometrically. Analysis revealed that both the amidated brevinin-1 peptides at their MICs could depolarize the membranes of S. aureus and V. cholerae. This was indicated by a marked shift in the fluorescence peak of voltage sensitive dye to the right from the negative control. In the present study it was shown that both the peptides are capable of inducing membrane depolarization before permeabilization.

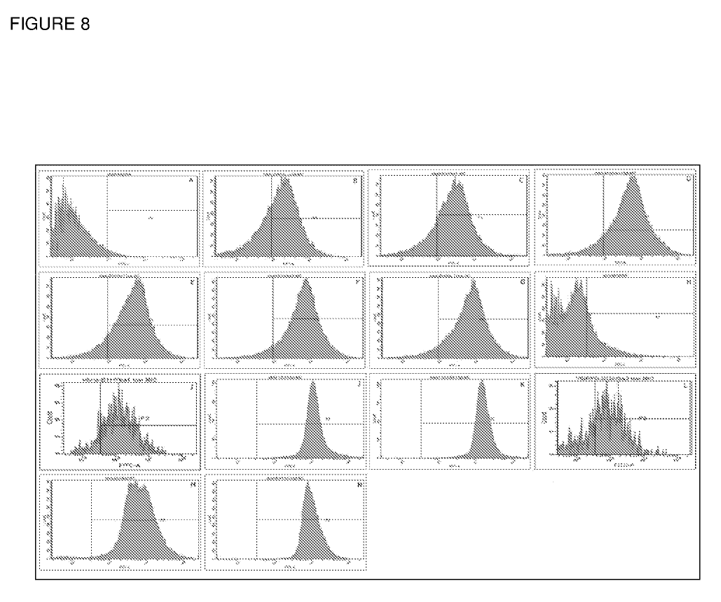

FIGS. 8A-8N are images of the evaluation of peptide concentration-dependent bacterial membrane damage. Concentration dependent SYTOX green uptake: S. aureus (FIGS. 8B-8G) and V. cholerae (FIGS. 8I-8N) were incubated with amidated B1 HYba1 (FIG. 8B: 0.5 μM, FIG. 8C: 1.5 μM, FIG. 8D: 8 μM, FIG. 8I: 2 μM, FIG. 8J: 12.5 μM, FIG. 8K: 25 μM) and amidated B1 HYba2 (FIG. 8E: 0.5 μM, FIG. 8F: 2.5 μM, FIG. 8G: 8 μM, FIG. 8L: 2 μM, FIG. 8M: 12 μM, FIG. 8N: 25 μM) for 15 minutes and was subjected to flow cytometric analysis. FIGS. 8A and 8H represent untreated controls. Difference in SYTOX green uptake was evaluated by the shift in fluorescence peak. Three different concentrations (sub-MIC, MIC and supra-MIC) of both the amidated peptides were used against S. aureus and V. cholerae. This was designed to analyze whether the peptides permeabilise the bacterial membrane in a concentration-dependent manner. Flow cytometric analysis was done using DNA binding dye SYTOX green. The fluorescent peaks showed a gradual shift from left to the right side of the graph (FIG. 8). This shift indicates increased SYTOX green uptake as concentration of the peptide increases. An interesting observation was the detection of SYTOX green signal at sub-MIC of the peptide, which is an indication of membrane rupture. It was thought that sub-MIC does not have any effect on bacteria and it grows more or less like the control, as evidenced from killing kinetics graph (FIG. 2). At this low concentration, pores might have formed which do not result in bacterial killing.

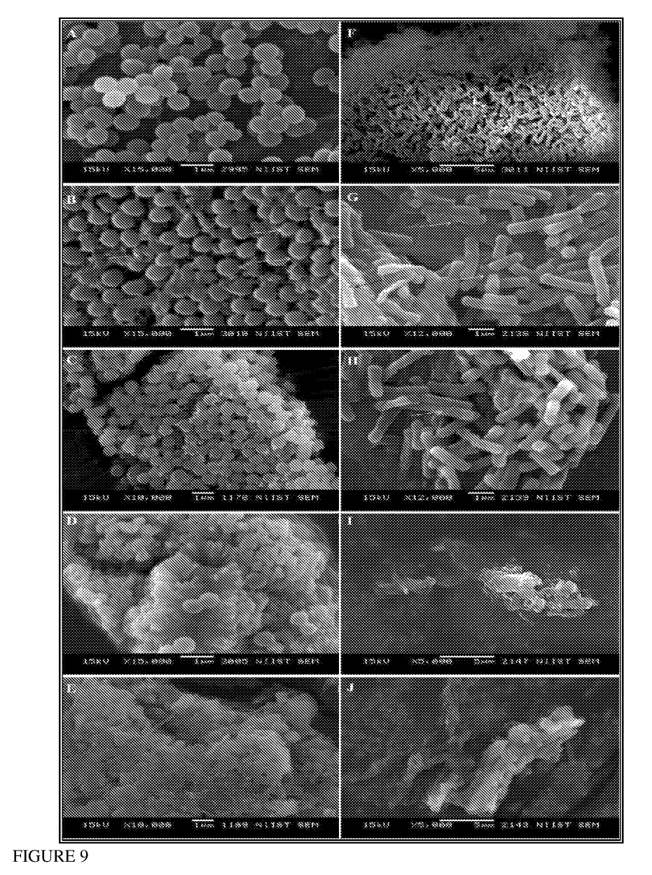

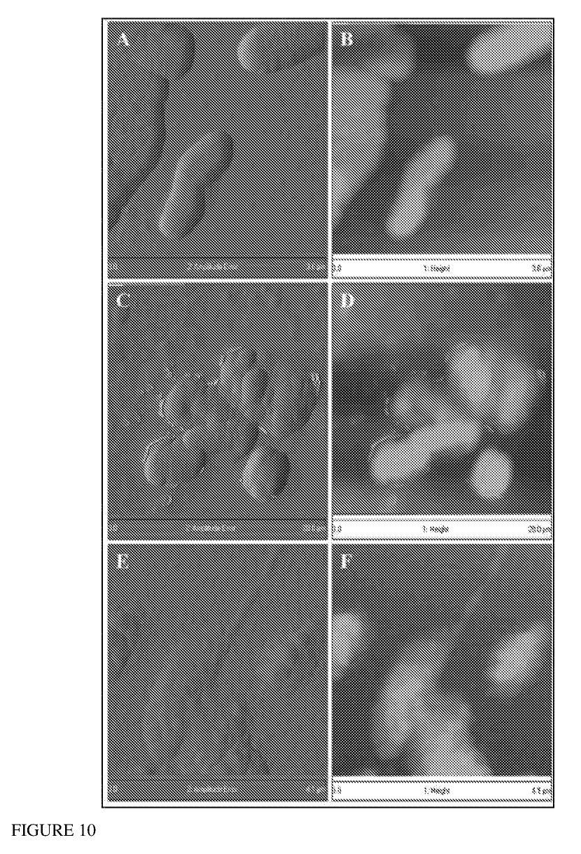

FIGS. 9A-9J are scanning electron microscopy images visualizing the changes in surface morphology of bacteria. SEM micrographs of FIG. 9A shows untreated S. aureus (round & intact). FIG. 9F shows V. cholerae (comma shaped & intact). S. aureus cells treated with MIC of B1HYba1 and B1 HYba2 for 10 minutes are shown in FIGS. 9B-9C and 15 minutes shown in FIGS. 9D-9E respectively. V. cholerae cells treated with MIC of B1HYba1 and B1HYba2 for 10 minutes (FIG. 9G-9H) and 15 minutes (FIG. 9I-9J) respectively. SEM analysis was done to gain more insights into the mechanism of action of B1/1 CONH₂ and B1/2 CONH₂. Extensive membrane damage of amidated brevinin-1 peptides treated S. aureus and V. cholerae were compared with the intact membrane of the control bacterium. Cells at two-time intervals of incubation (10 minutes and 15 minutes) were analyzed to visualize the changes at these time points. In the case of S. aureus, 10 minutes of incubation with both the peptides (FIGS. 9 B and C) showed intact cells with minor surface changes: Appearance of thread-like structures and debris were visible. S. aureus cells after 15 minutes incubation with both the amidated peptides exhibited complete destruction and aggregation: no intact cells were visible, ‘ghost-like’ structures were seen (FIGS. 9 D and 9E). Amidated brevinin-1 treated V. cholerae exhibited distinct morphological changes compared to control (FIGS. 9F-9J). Eeven though the cells were intact, after 10 minutes of incubation, they lost their characteristic comma shape. (FIGS. 9 G and H). After 15 minutes of incubation, aggregation and large clumps of ‘ghost cells’ were observed for both the peptides (FIGS. 91 and 9J). Visible damage was a confirmation of membrane disruption caused by the peptides.

FIGS. 10A-10F are atomic force microscopy images visualizing the changes in surface morphology of bacteria. FIGS. 10A and 10B are AFM images of untreated V. cholerae (MCV09). FIG. 10C-10F are AFM images of B1 HYba1 and B1 HYba2 treated V. cholerae cells respectively. V. cholerae is a comma-shaped, gram-negative bacterium. The control cells were having the characteristic shape with more or less smooth surface (FIG. 10A). The exposure to the B1/1 CONH₂ and B1/2 CONH₂ resulted in the changes in surface morphology and aggregation (FIGS. 10C and 10E) which were also observed in SEM analysis. The overall shape of the cells was lost and they became swollen, losing their characteristic shape.

A handful of peptides were characterized from Asian frogs but the most diverse Western Ghats remains untouched, with reports only from four frogs [28]. The rich biodiversity of the region might have influenced the evolution of various frog skin peptides against a diverse microbial population in the environment. This makes the peptide characterization from the region being the need of the hour. Only 5 families of peptides are reported from these frogs so far. They are brevinin-1 and brevinin-2 from Indosylvirana temporalis [30] and Clinotarsus curtipes [29], Hylaranakinin and esculentin 2 from Indosylvirana temporalis [31, 32] and tigerinins from Hoplobatrachus tigerinus [34]. These peptides were reported to be more potent than their analogs reported from other regions [45, 47]. Besides these, brevinin-1 from C. curtipes was reported to be potent against Mycobacterium tuberculosis and cancer cell lines [48].

We deduced the peptide sequence from the skin secretions of H. bahuvistara. The holocrine mode of secretion resulting in the release of intact poly-adenylated mRNAs and all cytoplasmic components made it possible to deduce its complete primary sequence which is not usually possible in MS [49]. The main advantages of this technique are the very low amount of sample requirement, noninvasiveness and being completely harmless to the sample donor [50]. The sample from few specimens would be sufficient for cloning whereas skin secretions from a large number of specimens are required for HPLC purification followed by MS analysis [51]. Tropical frogs have a low yield of skin secretion when compared to their temperate relatives hence mRNA cloning would be the best choice [28]. The Greater number of peptide sequences can be characterized by this method, as most of the mRNA could be cloned and sequenced [50].

Analysis of the cDNA library obtained from the lyophilized skin secretions of H. bahuvistara confirmed the presence of two biosynthetic host defense peptide precursors belonging to the brevinin-1 family. The 71 amino acid precursor exhibited analogous structural organization found in amphibian skin peptides with a highly conserved N-terminal signal region, an acid spacer that terminates in dibasic cleavage site KR and a highly variable C-terminal mature peptide. The mature peptides showed high sequence similarity to brevinin-1 SN1 characterized from the Chinese frog Sylvirana spinulosa [45]. Brevinins are among the ubiquitous linear, amphipathic and cationic antibacterial peptides, which consist of two families: Brevinin-1 (24 residues) and brevinin-2 (33-34 residues). The first members of the brevinin family were isolated from the frog Rana brevipoda porsa (renamed as Pelophylax porosus) and hence, the name [52]. The characteristic features shared by brevinin peptides are the presence of C-terminal disulfide-bridged cyclic heptapeptide (Cys18-Cys24), also called as Rana box [53]. This sequence is thought to play a critical role for its biological activity. The conserved amino acid sequences of brevinin-1 also include Ala9, Pro14, Cys18 and Cys24 [54]. Pro14 produces a stable kink in the molecule that stabilizes its structure [55]. Brevinin-1 exists predominantly as a random coil in aqueous solution but adopts an amphipathic α-helical structure in a hydrophobic membrane-mimetic environment [56]. The two paralogs characterized in this study revealed all the features described above and where matching with brevinin-1 peptides derived from shotgun cloning. The mature peptide sequences of brevinin-1 HYba1 and brevinin-1HYba 2 were 99% similar and having with a variable amino acid residue at 10th position.

Post-translational modifications (PTMs) are structural motifs invested on peptide families that are required for the biological function [50]. These modifications are added to the natural peptide sequence to confer desired functions (stability and increased activity) to the peptides [50]. Comparing the biological activities of natural and chemically modified peptides is useful in determining the effect of modifications. About 13 such modifications were reported in the literature [57]. Carboxy-terminal amidation and disulfide bridges are two such modifications which are thought to increase activity and stability. Enzymes that catalyze PTMs were also characterized from the anuran skin secretions [58, 59]. Two types of analogs for each peptide were designed and synthesized in the present study to analyze the effect of these modifications. The properties that are known to be important for their action such as charge and disulfide bond were modified/introduced. The net positive charge of the peptides was increased by C-terminal amidation, it was earlier reported that increasing the positive charge leads to increased membrane selectivity by enhancing the electrostatic interaction between the anionic membranes and peptides [27, 60]. The second type of analog was designed to incorporate two modifications, C-terminal amidation and a disulfide bond between C18 and C24 in both the peptides. S—S bonds are reported to be crucial in the activity of peptides because it stabilises their structure [61]. The reduction of antimicrobial activity due to disulfide bond modification was reported for brevinin-1, esculentinl, gaegurin 4 and ranalexin peptides [48, 62-65]. In this study, B1/1 COOH and B1/2 COOH exhibit activity only on selected gram-positive bacteria and not with gram-negative bacteria. On amidation, the peptides gained activity against both gram-positive and gram-negative bacteria by increasing the net charge and thereby increasing the biological activity, which is in agreement with the previous reports [66-68]. Upon amidation, the MIC value of B1/1 CONH₂ decreased from 9.5 μM to 1.5 μM for S. aureus, and for MRSA, the decrease was from 25 μM to 2.5 μM. For B1/2 CONH₂ the decrease was from 12.5 μM to 2.5 μM for S. aureus and 30 μM to 5 μM for MRSA.

Apart from increasing activity, C-terminal amidation is expected to increase the structural stability of the peptides, which permits strong interaction with the lipid moieties of the membrane [69] and lower the susceptibility to endopeptidase action [60]. These results clearly indicate the advantages of modifying the peptides to increase its activity. It is also suggested that a charged terminus destroys the antimicrobial activity [70]. These low MIC values against the harmful microbes indicate the therapeutic potential of these peptides. Strong inhibitory activity against MRSA and VRE may be a proof of the competence of HDPs against the multidrug resistant pathogens. Comparison of killing kinetics of S. aureus also reflects the effect of amidation, where the time taken for complete elimination of the bacterial population was reduced significantly. Studies with amidated PMAP 23 [63] found that they align perpendicular to the microbial membrane in contrast to the parallel alignment exhibited by non-amidated form. The difference in positioning on the membrane is thought to influence its activity [71]. Future studies would reveal whether the isolated brevinin-1 peptides show the same structural difference as PMAP 23. Incorporation of disulfide bridge did not significantly affect the MIC of gram-positive and negative bacteria (except for fish pathogens). These observations were in good accordance with the studies with peptides from C. curtipes [48] and Glandirana emeljanovi [72].

Evaluation of antimicrobial activity against gram-negative fish pathogens (A. sobria and A. hydrophila) demonstrated that amidated and cyclic peptides were active against both the pathogens while natural peptides were inactive at the tested concentration. Comparing the MICs of amidated and cyclic peptides, a fourfold decrease in the MIC was observed for cyclic brevinin-1 analogs against A. sobria. For A. hydrophilia, a significant decrease in MICs was not observed.

Comparing the hemolytic activity of natural, amidated and cyclic peptides, it was found that there was no significant change in hemolysis. This is advantageous because these modifications especially amidation increased the activity without increasing the hemolytic activity. It was previously reported that C-terminal amidation increases peptide activity and increases its hemolytic effect [69, 73], which is inconsistent with our results. Our results go in hand with recent findings in hemolysis of amidated brevinin-1 from C. curtipes, another endemic frog species of Western Ghats where the percentage hemolysis is below 40% [29, 48, 70].

The cation displacement assay was designed in such a way to get insights into the mechanism of action of the peptides under study. The changes in the activity of both the amidated peptides were evaluated in the presence of 20 mM Mg²⁺ and Ca²⁺ ions. These ions have binding sites on membrane lipopolysaccharides of gram-negative bacteria, which is expected to have a role in peptide action [42, 74]. The high concentration of both the metal ions abolished the activity of both the peptides against V. cholerae. Most of the antimicrobial peptides were reported to be salt sensitive, reduce or lose their activity in the presence of cations. They include α-defensins HD-5, β-defensins [75, 76], s-thanatin [42], melmine [41] and human cathelicidin LL-37 [77]. All these results point to the fact that these antagonisms were the result of a competitive inhibition. Cationic peptides displace these ions from their binding sites on the bacterial membrane during membrane permeabilization. When the same was evaluated against gram-positive S. aureus, the activity of the peptides was reduced but retaining their activity. This indicates that these peptides kill gram-positive bacteria without interaction with ions. This was a deviation from already reported thanatin and s-thanatin peptides which lost activity against B. subtilis at high salt concentrations [42]. It could be speculated that these cationic peptides interact with gram-negative bacterial membrane via cation binding sites and mediate lysis, [41] but for gram-positive bacteria peptide-membrane interaction follows a mechanism independent of cation binding sites.

As both the isolated peptides have same structural features of already reported brevinin-1 peptides [30, 45, 52], they are thought to have similar structural features. Analysis of the primary sequence of both the peptides, it was found that they are helical, and their amphipathic nature was revealed by helical wheel plots (FIG. 1a). Helical wheel prediction of the amphipathic α-helical structure was also reported for brevinin 1BYa [78], magainin and melittin [79]. Amphipathic nature of the peptide is a prerequisite for membrane action [25, 80]. The segregation of hydrophobic residues to one side of the helix enables interaction with the lipid heads on the membrane and their deep insertion into the interior of the membrane. Brevinin-1 peptides exist as random coil in the aqueous environment and helical in membrane mimetic environment. The CD analysis performed in the present study confirms this and is in agreement with similar reports on the secondary structure of brevinin-1 peptides in different environments [81, 82].

Most of the AMPs are reported to be membrane active and their exact mechanism of action is still unclear [10]. Based on fluorescent probes, SEM analysis, AFM analysis and NMR studies, various models have been proposed [44, 83-85]. The models include barrel-stave model, where the peptides induce a voltage-dependent channel formed by the aggregation of peptide monomers on the membrane surface followed by insertion into the membrane to form a pore [86]. The carpet model explains membrane disruption due to parallel alignment of peptides on the membrane creating a transient ‘toroidal pore’ followed by permeation/disruption of these membranes [87, 88]. On the outer membrane of gram-negative bacteria, the peptide inserts and translocate via ‘self-promoted uptake’. On reaching the anionic inner membrane, electrostatic and hydrophobic interactions guide permeation/disruption of these membranes [89]. The present study addresses the question whether B1/1 CONH₂ and B1/2 CONH₂ act on the bacterial membrane via classical pathways or not. To address this, we performed fluorescent probing along with SEM and AFM in order to monitor the changes in the bacterial structure. Confocal analysis revealed that SYTOX green enters the bacterial cells with altered membrane permeability. Flow cytometric analysis with the same probe and three different peptide concentrations show that membrane disruption does not always lead to cell death. Interestingly, at sub-MIC, where the bacteria were believed to grow more or less similar to control bacteria, SYTOX green signal was obtained for both the peptides in both the tested organisms. The same was observed for temporin L, and suggested that this property, i.e., causing membrane permeation without killing the cell, could be utilized for designing ‘helper agents’ [83]. These agents could be used for combinatorial therapy, where they permeabilized the cells so that impermeable drugs can enter and kill the bacteria.

Results of flow cytometric analysis demonstrated that the extent of membrane disruption increases with increasing peptide concentration and leads to bacterial death through significant disruption of the membrane structure. These results are in agreement with ultrashort antibacterial and anti-fungal peptides which showed the same effect on both gram-positive and negative bacteria and fungi [44]. Studies with voltage-sensitive dye DiBAC4(3) revealed that both the peptides cause membrane depolarization of S. aureus and V. cholerae before killing. Peptide-induced membrane permeabilization is preceded by the change in the membrane potential; every peptide does cause this depolarization [29]. Membrane depolarization effect was also reported for brevinin-1 identified from C. curtipes [29,48].

SEM analysis of S. aureus and V. cholerae revealed that these peptides lyse the bacteria in a distinct pattern without blebbing. Previous reports on temporin L also confirms the same [83]. After 10 minutes of incubation, the peptides initiated pore formation without changes in the morphology of the bacteria, which could be identified from the thread like structures and debris [90, 91]. Incubating the bacteria for 15 minutes caused complete disruption of the cells, leaving ‘ghost-like’ structures formed as a result of aggregation [81, 82]. These results confirm the killing kinetics data which revealed 100% killing of both the bacteria by amidated peptides in 15 minutes. In the case of V. cholerae, there was a significant change in cell shape on incubation at MIC for 15 minutes of incubation. The abnormalities observed include loss of comma shape, the fusion of adjacent cells and ghost-like appearance with cellular debris. These results go in hand with previously reported CEMA [92], sarcotoxin I [93], hecate-1 [94], melittin [94], PGYa [95], SMAP-29 [96], and magainin peptides [91]. This observation was also consistent with the observations made on E. coli cells under PGLa stress [69]. AFM results further confirm the findings of SEM observations. AFM analysis of V. cholerae with both the peptides at MIC showed roughening of the surface compared to control and appearance of ghost-like structures and fused cells as previously reported [84, 97-99]

Killing of both gram-positive and negative bacteria by brevinin-1 and its analogs identified from the skin secretion of H. bahuvistara has been shown. Initial peptide-bacteria interaction causes depolarization of the bacterial membrane followed by pore formation with the appearance of cellular debris and thread-like structures, which terminates in aggregation and clumping of cells which resembles ‘ghost-like structures’.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Siano A, Gatti P I, Imaz M S, Zerbini E, Simonetta A C, Lajmanovich R and Tonarelli G G. A Comparative Study of the Biological Activity of Skin and Granular Gland Secretions of Leptodactylus latrans and Hypsiboas pulchellus from Argentina. Rec Nat Prod. 2014; 8: 128-135.
2. Albericio F and Kruger H G. Therapeutic peptides. Future Med Chem. 2012; 4: 1527-1531.
3. Kaspar A A, Reichert J M. Future directions for peptide therapeutics development. Drug Discov Today. 2013; 18: 807-817.
4. Xu X, Lai R. The chemistry and biological activities of peptides from amphibian skin secretions. Chem Rev. 2015; 115(4): 1760-846.
5. Nicolas P. Multifunctional host defense peptides: intracellular-targeting antimicrobial peptides. FEBS J. 2009; 276(22): 6483-6496.
6. Laverty G, Gorman S P, and Gilmore B F. The Potential of Antimicrobial Peptides as Biocides. Int J Mol Sci. 2011; 12(10): 6566-659.
7. Koniga E, Bininda-Emonds O R P, Shaw C. The diversity and evolution of anuran skin peptides. Peptides. 2015; 63: 96-117.
8. Aoki W, Kuroda K, Ueda M. Next generation of antimicrobial peptides as molecular targeted medicines. Journal of Bioscience and Bioengineering. 2012; 114(4): 365-370.
9. Rahnamaeian M. Antimicrobial peptides Modes of mechanism, modulation of defense responses. Plant Signaling and Behavior. 2011; 6: 1325-1332.
10. Nakatsuji T, Gallo R L. Antimicrobial Peptides: Old Molecules with New Ideas. Journal of Investigative Dermatology. 2011; 132: 887-895.
11. Silva F P, Machado M C C. Antimicrobial peptides: Clinical relevance and therapeutic implications. Peptides. 2012; 36: 308-314.
12. Tokumaru S, Sayama K, Shirakata Y, Komatsuzawa H, Ouhara K, Hanakawa Y, Yahata Y, Dai X, Tohyama M, Nagai H, Yang L, Higashiyama S, Yoshimura A, Sugai M, Hashimoto K. Induction of keratinocyte migration via transactivation of the epidermal growth factor receptor by the antimicrobial peptide LL-37. J Immunol. 2005; 175: 4662-4668.
13. Dodd G T, Mancini G, Lutz B, Luckman S M. The peptide hemopressin acts through CB1 cannabinoid receptors to reduce food intake in rats and mice. J Neurosci. 2010; 30: 7369-7376.
14. Scott M G, Davidson D J, Gold M R, Bowdish D, Hancock R E. The human antimicrobial peptide LL-37 is a multifunctional modulator of innate immune responses. J Immunol. 2002; 169: 3883-3891.

15. Mulder K C, Lima L A, Miranda V J, Dias S C, Franco O L. Current scenario of peptide-based drugs: the key roles of cationic antitumor and antiviral peptides. Front Microbiol. 2013; 4: 321.
16. Fritz J H, Brunner S, Birnstiel M L, Buschle M, Gabain A, Mattner F, Zauner W. The artificial antimicrobial peptide KLKLLLLLKLK induces predominantly a TH2-type immune response to co-injected antigens. Vaccine. 2004; 22: 3274-3284.
17. Zasloff M. Antimicrobial peptides of multicellular organisms. Nature. 2002; 415: 389-395.
18. Brakch N, Allemandou F, Cavadas C, Grouzmann E and Brunner H R. Dibasic cleavage site is required for sorting to the regulated secretary pathway for both pro- and neuropeptide Y. Journal of Neurochemistry. 2002; 81: 1166-1175.
19. Hancock R E, Sahl H G. Antimicrobial and host-defense peptides as new anti-infective therapeutic strategies. Nat. Biotechnol. 2006; 24: 1551-1557.
20. Peravalil J B, SrKotra, Sobha K, Nelson R, Rajesh K V, Pulicherla K K. Antimicrobial Peptides: An Effective Alternative for Antibiotic Therapy. Mintage Journal of Pharmaceutical and Medical Sciences. 2013; 1-7.
21. Huang H W. Molecular mechanism of antimicrobial peptides: The origin of cooperativity. Biochimica et Biophysica Acta. 2006; 1758: 1292-302.
22. Pasupuleti M, Schmidtchen A, Malmsten M. Antimicrobial peptides: key components of the innate immune system. Critical Reviews in Biotechnology. 2011; 1-29.
23. Fjell C D, Hiss J A, Hancock R E W, Schneider G. Designing antimicrobial peptides: form follows function. Nature Reviews, Drug Discovery. 2012; 11: 37-57.
24. Lazrev V N, Govorum V M. Antimicrobial peptides and their uses in medicine. Applied Biochemistry and Microbiology. 2010; 46: 803-814.
25. Tossi A, Sandri L, Giangaspero A. Amphipathic, alpha-helical antimicrobial peptides. Biopolymers. 2000; 55: 4-30.
26. Zelezetsky I, Tossi A. Alpha-helical antimicrobial peptides-Using a sequence template to guide structure-activity relationship studies. Biochimica et Biophysica Acta. 2006; 1758: 1436-1449.
27. Bahar A A, Ren D. Antimicrobial Peptides. Pharmaceuticals. 2013; 6: 1543-1575.
28. Vineeth Kumar T V, Holthausen D, Jacob J, George S. Host Defense Peptides from Asian Frogs as Potential Clinical Therapies. Antibiotics. 2015; 4: 136-159.
29. Abraham P, George S, Santhosh Kumar K. Novel antibacterial peptides from the skin secretion of the Indian bicoloured frog Clinotarsus curtipes. Biochimie. 2013; 97: 144-151
30. Reshmy V, Preeji V, Abraham P, Santhosh Kumar K, George S. Three novel antimicrobial peptides from the skin of the Indian bronzed frog Hylarana temporalis (Anura: Ranidae). J. Pept. Sci. 2011; 17: 342-347.
31. Reshmy V, Santhosh Kumar K, George S. Full length cDNA derived novel peptides belonging to Esculentin Family from skin of Indian Bronzed Frog Hylarana temporalis. Res. J. Biotech. 2011; 6: 71-74.
32. Reshmy V, Preeji V, Parvin A, Santhosh Kumar K, George S. Molecular cloning of a novel Bradykinin-related peptide from the skin of Indian bronzed frog Hylarana temporalis. J. Genomics Insights. 2010; 3: 23-28.
33. Ramesh B, Vadivelu P, Kavitha K, Suresh G, Ravichandran N, Siva G V. Antimicrobial peptide from Euphlyctis hexadactylus and its efficacy against plant pathogens. Int. J. Curr. Res. 2010; 6: 14-17.
34. Sai K P, Jagannadham M V, Vairamani M, Raju N P, Devi A S, Nagaraj R, Sitaram N. Tigerinins: Novel antimicrobial peptides from the Indian frog Rana tigerina. J. Biol. Chem. 2001; 276: 2701-2707.
35. Padhye A D, Jadhav A, Modak N, Nameer P O, Dahanukar N. Hydrophylax bahuvistara, a new species of fungoid frog (Amphibia: Ranidae) from peninsular India. Journal of Threatened Taxa. 2015; 7(11): 7744-7760.
36. Tyler M J, Stone D J, Bowie J H. A novel method for the release and collection of dermal, glandular secretions from the skin of frogs. J Pharmacol Toxicol Methods. 1992; 28: 199-200.
37. Drozdetskiy A, Cole C, Procter J, Barton G J. JPred4: a protein secondary structure prediction server, Nucleic Acids Res. 2015
38. Jones D T. Protein Secondary Structure Prediction Based on Position-specific Scoring Matrices. J Mol Biol. 1999; 292: 195-202.
39. Donald S R, Thornsberry C. Broth-dilution method for determining the antibiotic susceptibility of anaerobic bacteria. Antimicrobial agents and chemotherapy. 1975; 7: 15-21.
40. Nielsen S L, Frimodt-Moller N, Kragelund B B, Hansen P R. Structure activity study of the antibacterial peptide fallaxin. Protein Sci. 2007; 16: 1969-1976.
41. Rasul R, Cole N, Balasubramanian D, Chen R, Kumar N, Willcox M D P. Interaction of the antimicrobial peptide melimine with bacterial membranes. International Journal of Antimicrobial Agents. 2010; 35(6): 566-572.
42. Wu G, Ding J, Li H, Li L, Zhao R, Fan X, Shen Z. Effects of Cations and P H on Antimicrobial Activity of Thanatin and s-Thanatin against Escherichia coli ATCC25922 and Bacillus subtilis ATCC 21332. Current Microbiology. 2008; 57(6): 552-557.
43. Nuding S, Fellermann K, Wehkamp J, Mueller H A, Stange E F. A flow cytometric assay to monitor antimicrobial activity of defensins and cationic tissue extracts. J. Microbiol. Methods. 2006; 65: 335-345.
44. Makovitzki A, Avrahami D, Shai Y. Ultrashort Antibacterial and Antifungal Lipopeptides. PNAS. 2006; 103(43): 15997-16002.
45. Yang X, Hu Y, Xu S, Hu Y, Meng H, Guo C, Liu Y, Liu J, Yu Z, Wang H. Identification of multiple antimicrobial peptides from the skin of fine-spined frog, Hylarana spinulosa (Ranidae). Biochimie. 2013; 95: 2429-2436.
46. Thomas P, Vineeth Kumar T V, Reshmy V, Santhosh Kumar K, George S. A mini review on the antimicrobial peptides isolated from the genus Hylarana (Amphibia: Anura) with a proposed nomenclature for amphibian skin peptides. Mol. Biol. Rep. 2012; 39: 6943-6947.
47. Conlon M J, Kolodziejek J, Nowotny N, Leprince J, Vaudry H, Coquet L, Jouenne T, King J D. Characterization of antimicrobial peptides from the skin secretions of the Malaysian frogs, Odorrana hosii and Hylarana picturata (Anura:Ranidae). Toxicon. 2008; 52: 465-473.
48. Abraham P, Sundaram A, Asha R, Reshmy V, George S, Kumar K S. Structure-Activity Relationship and Mode of Action of a Frog Secreted Antibacterial Peptide B1CTcu5 Using Synthetically and Modularly Modified or Deleted (SMMD) Peptides. PLoS ONE. 2015; 10(5): 1-14.
49. Chen T, Farragher S, Bjourson A J, Orr D F, Rao P, Shaw C. Granular gland transcriptomes in stimulated amphibian skin secretions. Biochem J. 2003; 371: 125-130.
50. Pinksea M, Evaristoa G, Pietersea M, Yua Y, Verhaerta P. M S approaches to select peptides with post-translational modifications from amphibian defense secretions prior to full sequence elucidation. eupa open proteomics. 2014; 5: 32-40.
51. Roelants K, Fry B G, Norman J A, Clynen E, Schoofs L, Bossuyt F. Identical skin toxins by convergent molecular adaptation in frogs. Curr Biol. 2010; 20: 125-130.
52. Morikawa N, Hagiwari K, Nakajima T. Brevinin 1 and 2, unique antimicrobial peptides from skin of the frog Rana brevipoda porsa. Biochem. Biophys. Res. Commun. 1992; 189: 184-190.
53. Conlon J M, Sonnevend A, Jouenne T, Coquet L, Cosquer D, Vaudry H, Iwamuro S. A family of acyclic brevinin-1 peptides from the skin of the Ryukyu brown frog Rana okinavana. Peptides. 2005; 26(2): 185-190.
54. Conlon J M, Kolodziejek J, Nowotny N. Antimicrobial peptides from ranid frogs: taxonomic and phylogenetic markers and a potential source of new therapeutic agents. Biochim Biophys Acta. 2004; 1696(1): 1-14.
55. Suh J Y, Lee K H, Chi S W, Hong S Y, Choi B W, Moon H M, Choi B S. Unusually stable helical kink in the antimicrobial peptide derivative of gaegurin. FEBS Lett. 1996; 392(3): 309-312.
56. Kwon M Y, Hong S Y, Lee K H. Structure-activity analysis of brevinin 1E amide, an antimicrobial peptide from Rana esculenta. Biochim Biophys Acta. 1998; 1387(1-2): 239-248.
57. Wang G. Post-translational Modifications of Natural Antimicrobial Peptides and Strategies for Peptide Engineering. Curr Biotechnol. 2014; 1(1): 72-79.
58. Mizuno K, Ohsuye K, Wada Y, Fuchimura K, Tanaka S, Matsuo H. Cloning and sequence of cDNA encoding a peptide C-terminal-amidating enzyme from Xenopus laevis. Biochem Biophys Res Commun. 1987; 148: 546-552.
59. Mollay C, Wichta J, Kreil G. Detection and partial characterization of an amidating enzyme in skin secretion of Xenopus laevis. FEBS Lett. 1986; 202: 251-254.
60. Rivas L, Luque-Ortega J R, Andreu D. Amphibian antimicrobial peptides and Protozoa: Lessons from parasites. Biochimica et Biophysica Acta. 2009; 1788: 1570-1581.
61. Nolde S B, Vassilevski A A, Rogozhin E A, Barinov N A, Balashova T A, Samsonova O V, Baranov Y V, Feofanov A V, Egorov T A, Arseniev A S. Disulfide-stabilized helical hairpin structure and activity of a novel antifungal peptide EcANP1 from seeds of barnyard grass (Echinochloa crusgalli). J Biol Chem. 2011; 286: 25145-25153.
62. Haney E F, Hunter H N, Matsuzaki K, Vogel H J. Solution NMR studies of amphibian antimicrobial peptides: linking structure to function? Biochimica et Biophysica Acta (BBA)-Biomembranes. 2009; 1788: 1639-1655.
63. Kim H, Kim S, Lee M, Lee B, Ryu P. Role of C-terminal heptapeptide in pore-forming activity of antimicrobial agent, gaegurin 4. The Journal of Peptide Research. 2004; 64: 151-158.
64. Mangoni M L, Fiocco D, Mignogna G, Barra D, Simmaco M. Functional characterisation of the 1-18 fragment of esculentin-1b, an antimicrobial peptide from Rana esculenta. Peptides. 2003; 24: 1771-1777.
65. Vignal E, Chavanieu A, Roch P, Chiche L, Grassy G, Calas B, Aumelas A. Solution structure of the antimicrobial peptide ranalexin and a study of its interaction with perdeuterated dodecylphosphocholine micelles. Eur J Biochem. 1998; 253: 221-228.
66. Feder R, Nehushtai R, Mor A. Affinity driven molecular transfer from erythrocyte membrane to target cells. Peptides. 2001; 22: 1683-1690.
67. Shalev D E, Mor A, Kustanovich I. Structural consequences of carboxyamidation of dermaseptin S3. Biochemistry. 2002; 41: 7312-7317.
68. Mor A, Hani K, Nicolas P. The vertebrate peptide antibiotics dermaseptins have overlapping structural features but target specific microorganisms, J. Biol. Chem. 1994; 269: 31635-31641.
69. da Silva A V, De Souza B M, Dos Santos Cabrera M P, Dias N B, Gomes P C, Neto J R, Stabeli R G, Palma M S. The effects of the C-terminal amidation of mastoparans on their biological actions and interactions with membrane-mimetic systems. Biochimica et Biophysica Acta. 2014; 1838: 2357-2368.
70. Strandberg E, Tiltak D, leronimo M, Kanithasen N, Wadhwani P, Ulrich A S. Influence of C-terminal amidation on the antimicrobial and hemolytic activities of cationic-helical peptides. Pure Appl. Chem. 2007; 79(4): 717-728.
71. Kim J Y, Park S C, Yoon M Y, Hahm K S, Park Y. C-terminal amidation of PMAP-23: translocation to the inner membrane of Gram-negative bacteria. Amino Acids. 2011; 40(1): 183-195.
72. Won H S, Kang S, Lee B. Action mechanism and structural requirements of the antimicrobial peptides, gaegurins. Biochimica et Biophysica Acta. 2009; 1788: 1620-1629.
73. Chen H, Brown J H, Morell J L, Huang C M. Synthetic magainin analogs with improved antimicrobial activity. 1988; 236 (2): 462-466.
74. Hancock R E, Karunarame D N, Bernegger-Egli C. In Bacterial Cell Wall. Ghuysen J M, Hakenbeck R, eds. 1994; 263-279.
75. Tomita T, Hitomi S, Nagase T, Matsui H, Matsuse T, Kimura S, Ouchi Y. Effect of ions on antibacterial activity of human beta defensin 2. Microbiol Immunol. 2000; 44: 749-754.
76. Hoover D M, Wu Z, Tucker K, Lu W, Lubkowski J. Antimicrobial characterization of human beta-defensin 3 derivatives. Antimicrob Agents Chemother. 2003; 47: 2804-2809.
77. Cox D L, Sun Y, Liu H, Lehrer R I, Shafer W M. Susceptibility of Treponema pallidum to host-derived antimicrobial peptides. Peptides. 2003; 24: 1741-1746.
78. Conlon J M, Sonnevend A, Patel M, Davidson C, Nielsen P F, Pal T, Rollins-Smith L A. Isolation of peptides of the brevinin-1 family with potent candidacidal activity from the skin secretions of the frog Rana boylii. J. Peptide Res. 2003; 62: 207-213.
79. Dathe M, Wieprecht T. Structural features of helical antimicrobial peptides: their potential to modulate activity on model membranes and biological cells. Biochimica et Biophysica Acta. 1999; 1462: 71-87.
80. Harris F, Dennison S R, Phoenix D A. Anionic antimicrobial peptides from eukaryotic organisms. Curr Protein Pept Sci. 2009; 10: 585-606.
81. Sun Y, Dong W, Sun L, Ma L, Shang D. Insights into the membrane interaction mechanism and antibacterial properties of chensinin-1b. Biomaterials. 2015; 37: 299-311.
82. Joshi S, Bisht G S, Rawat D S, Kumar A, Kumar R, Maiti S, Pasha S. Interaction studies of novel cell selective antimicrobial peptides with model membranes and E. coli ATCC 11775. Biochimica et Biophysica Acta. 2010; 1798: 1864-1875.
83. Mangoni M L, Papo N, Barra D, SimmacoM, Bozzi A, Di Giulio A, Rinaldi A C. Effects of the antimicrobial peptide temporin L on cell morphology, membrane permeability and viability of Escherichia coli. Biochem J. 2004; 380: 859-865.
84. M. Meincken, Holroyd D L, Rautenbach M. Atomic Force Microscopy Study of the Effect of Antimicrobial Peptides on the Cell Envelope of Escherichia coli. Antimicrobial Agents and Chemotherapy. 2005; 49: 4085-4092.
85. Strandberg E, Zerweck J, Wadhwani P, Ulrich A S. Synergistic Insertion of Antimicrobial Magainin-Family Peptides in Membranes Depends on the Lipid Spontaneous Curvature. Biophysical Journal. 2013; 104: 09-11.
86. Ehrenstein G, Lecar H. Electrically gated ionic channels in lipid bilayers. Q Rev Biophys. 1977; 10:1-34.
87. Matsuzaki K, Murase O, Fujii N, Miyajima K. An antimicrobial peptide, magainin 2, induced rapid flip-flop of phospholipids coupled with pore formation and peptide translocation. Biochemistry. 1996; 35: 11361-11368.
88. BozelliJr J C, Sasahara E T, Pinto M R, Nakaie C R, Schreier S. Effect of head group and curvature on binding of the antimicrobial peptide tritrpticin to lipid membranes. Chem Phys Lipids. 2012; 165: 365-373.
89. Hancock R E, Chapple D S. Peptide Antibiotics. Antimicrobial Agents and Chemotherapy. 1999; 43: 1317-1323.
90. Hartmann M, Berditsch M, Hawecker J, Ardakani M F, Gerthsen D, Ulrich A S. Damage of the Bacterial Cell Envelope by Antimicrobial Peptides Gramicidin S and PGLa as Revealed by Transmission and Scanning Electron Microscopy. Antimicrobial Agents and Chemotherapy. 2010; 54(8): 3132-3142.
91. Matsuzaki K, Sugishit K, Harada M, Fujii N and Miyajima, K. Interactions of an antimicrobial peptide, magainin 2, with outer and inner membranes of Gram-negative bacteria. Biochim. Biophys. Acta. 1997; 1327: 119-130.
92. Hancock, R E. Cationic peptides: effectors in innate immunity and novel antimicrobials. Lancet Infect. Dis. 2001; 1: 156-164.
93. Okada M, Natori S. Mode of action of a bactericidal protein induced in the haemolymph of Sarcophaga peregrina (flesh-fly) larvae. Biochem J. 1984; 222: 119-124.
94. Henk W G, Todd W J, Enright F M, Mitchell P S. The morphological effects of two antimicrobial peptides, hecate-1 and melittin, on Escherichia coli. Scanning Microsc. 1995; 9: 501-507.
95. Tiozzo, E., Rocco, G., Tossi, A. and Romeo, D. Wide-spectrum antibiotic activity of synthetic, amphipathic peptides. Biochem. Biophys. Res. Commun. 1998; 249: 202-206.
96. Skerlavaj B., Benincasa M, Risso A, Zanetti M, Gennaro R. SMAP-29: a potent antibacterial and antifungal peptide from sheep leukocytes. FEBS Lett. 1999; 463: 58-62.
97. Eaton P, Fernandes J C, Pereira E, Pintado M E, Xavier Malcata F. Atomic force microscopy study of the antibacterial effects of chitosans on Escherichia coli and Staphylococcus aureus. Ultramicroscopy. 2008; 108(10): 1128-1134.
98. A. Li, P. Y. Lee, B. Ho, J. L. Ding, C. T. Lim. Atomic force microscopy study of the antimicrobial action of Sushi peptides on Gram-negative bacteria. Biochimica et Biophysica Acta. 2007; 1768: 411-418.
99. Zdybicka-Barabas A, Januszanis B, Mak P, Cytryńska M. An atomic force microscopy study of Galleria mellonella apolipophorin III effect on bacteria. Biochimica et Biophysica Acta. 2011; 1808: 1896-1906.

1. A cDNA composition encoding a peptide for reducing a bacterial population comprises: an isolated cDNA encoding a brevinin-1 HYba1 peptide, a brevinin-1 HYba2 peptide or both.

2. An antimicrobial composition for the treatment of a bacterium, wherein the composition comprises: a pharmaceutically effective amount of a modified brevinin-1 peptide disposed in a pharmaceutical carrier.

3. A modified brevinin-1 peptide composition for use as a medicament for the treatment of a bacterial infection wherein the composition comprises: a pharmaceutically effective amount of modified brevinin-1 peptide disposed in a pharmaceutical carrier.

4. A method of making a modified brevinin-1 peptide composition for use as a medicament for the treatment of a bacterial infection comprising the steps of:
providing a brevinin-1 peptide;
modifying the brevinin-1 peptide to contain a —COOH group or a —CONH₂ group to form a modified brevinin-1 peptide having at least 85% homology to SEQ ID NOS: 7-12; and
combining a pharmaceutically effective amount of the modified brevinin-1 peptide with a pharmaceutical carrier.

5. The cDNA composition encoding the brevinin-1 peptide of claim 1, wherein the cDNA is disposed in a vector.

6. The composition of claim 1, wherein the modified brevinin-1 peptide comprises a brevinin-1 HYba1 peptide having a sequence selected from SEQ ID NOS: 7-9, a brevinin-1 HYba2 peptide selected from SEQ ID NOS: 10-12 or both.

7. The composition of claim 1, wherein the brevinin-1 peptide is a modified brevinin-1 peptide that has at least 85% homology to any sequence selected from SEQ ID NOS: 7-12.

8. The composition of claim 7, wherein the modified brevinin-1 peptide has at least 85% homology to SEQ ID NO: 7 or 10.

9. The composition of claim 7, wherein the modified brevinin-1 peptide has at least 85% homology to SEQ ID NO: 8 or 11.

10. The composition of claim 7, wherein the at least 85% homology is 86, 87, 88, 89, 90, 91, 92, 93, 94, 95, 96, 97, 98, 99, 99.5, 99.8, or 100% homology.

11. The composition of claim 4, wherein the pharmaceutical carrier is a liposome, an ointment, a paste, a solution, a hydrogel, a gel, a petroleum carrier, a polymer, or a combination thereof.

12. An antimicrobial composition for the treatment of a bacterium, wherein the composition comprises:
a pharmaceutically effective amount of a first active agent and a modified brevinin-1 peptide disposed in a pharmaceutical carrier, wherein the modified brevinin-1 peptide comprises a brevinin-1 HYba1 peptide having a sequence selected from SEQ ID NOS: 7-9, a brevinin-1 HYba2 peptide selected from SEQ ID NOS: 10-12 or both.

13. The composition of claim 12, wherein the first active agent comprises amoxicillin, doxycycline, cephalexin, ciprofloxacin, clindamycin, metronidazole, azithromycin, sulfamethoxazole/trimethoprim, amoxicillin/clavulanate, levofloxacin, clotrimazole, econazole nitrate, miconazole, terbinafine, fluconazole, ketoconazole, or amphotericin.

14. The peptide of claim 4, wherein the modified brevinin-1 peptide comprises a brevinin-1 HYba1 peptide having a sequence selected from SEQ ID NOS: 7-9, a brevinin-1 HYba2 peptide selected from SEQ ID NOS: 10-12 or both.

15. The peptide of claim 4, wherein the modified brevinin-1 peptide has at least 85% homology to any sequence selected from SEQ ID NOS: 7-12.

16. The composition of claim 3, wherein the modified brevinin-1 peptide has at least 85% homology to SEQ ID NO: 7 or 10.

17. The peptide of claim 3, wherein the pharmaceutical carrier is a liposome, an ointment, a paste, a solution, a hydrogel, a gel, a petroleum carrier, a polymer, or a combination thereof.

18. The method of claim 4, wherein the modified brevinin-1 peptide comprises a brevinin-1 HYba1 peptide having a sequence selected from SEQ ID NOS: 7-9, a brevinin-1 HYba2 peptide selected from SEQ ID NOS: 10-12 or both.

19. The method of claim 4, wherein the modified brevinin-1 peptide has at least 85% homology to any sequence selected from SEQ ID NOS: 7-12.

20. A modified brevinin-1 peptide having at least 85% homology to SEQ ID NOS: 7-12; modified to comprise a —COOH group or a —CONH₂ group.
